(19) United States
(12) Patent Application Publication
(10) Pub. No.: US 2014/0275049 A1
(43) Pub. Date: Sep. 18, 2014

(54) SUBSTITUTED PYRAZOLE ANALOGS As RAR ANTAGONISTS

(71) Applicant: Eli Lilly and Company, Indianapolis, IN (US)

(72) Inventors: Thomas John Bleisch, Noblesville, IN (US); David Andrew Coates, Indianapolis, IN (US); Norman Earle Hughes, Indianapolis, IN (US); Scott Alan Jones, Indianapolis, IN (US); Bryan Hurst Norman, Indianapolis, IN (US)

(21) Appl. No.: 14/350,576

(22) PCT Filed: Oct. 19, 2012
(86) PCT No.: PCT/US2012/060995

§ 371 Date: Apr. 9, 2014

Related U.S. Application Data
(60) Provisional application No. 61/553,597, filed on Oct. 31, 2011.

(51) Int. Cl. C07D 403/10; C07D 401/10; A61K 31/454; A61K 31/4439; A61K 31/415; A61K 31/541; A61K 31/5377; C07D 401/04; A61K 31/496; C07D 231/12

(52) U.S. Cl. CPC C07D 403/10; A61K 31/496; C07D 401/10; A61K 31/454; C07D 231/12; A61K 31/415; A61K 31/541; A61K 31/5377; C07D 401/04; A61K 31/4439
USPC 514/227.8; 544/371; 514/254.05; 546/211; 514/326; 548/377.1; 514/406; 544/58.4; 544/140; 514/236.5; 546/275.4; 514/341

(57) ABSTRACT

The present invention provides compounds of Formula I or a pharmaceutical salt thereof; methods of treating osteoarthritis and the pain associated with osteoarthritis using the compounds; and processes for preparing the compounds.

[chemical structure]

Osteoarthritis is a complex degenerative disease characterized by progressive destruction of articular cartilage and peri-articular structures including bones, synovial, and associated fibrous joint tissues. Existing drug therapies can reduce pain associated with osteoarthritis, but over time become only moderately effective. Each of the current standard of care therapies has variable risk/benefit considerations. Individuals can become refractory to specific drug treatments and/or are contraindicated for the treatments due to pre-existing or emergent cardiovascular and/or gastric intestinal conditions. Consequently, there remains a need for additional treatment options to treat and alleviate pain from osteoarthritis.

Retinoids (including RAR agonists), are known to cause and/or exacerbate pain in animal models, demonstrate catabolic activity for cartilage, and induce osteoarthritis-like processes in animal models. Compounds which exhibit RAR antagonistic activity may provide an alternative treatment regime for patients suffering from osteoarthritis pain.

U.S. Pat. No. 5,464,178 discloses compounds including the compound below:

[chemical structure]

which is disclosed as being useful to treat pain associated with inflammation and arthritis. However the compounds are not described as exhibiting RAR gamma antagonism.

The present invention provides an alternative treatment for osteoarthritis, and in particular, an alternative treatment for the pain associated with osteoarthritis. The present invention may also address one or more deficiencies, such as, a reduction in the risks of undesired interactions with other drugs and the risk of pre-existing or emergent cardiovascular and/or gastric intestinal conditions under the current standard of care for osteoarthritis treatment regimes. Further, compounds of the present invention selectively bind to RARγ and may therefore provide advantages over non-selective RAR antagonists, which can be accompanied by a broad spectrum of toxic side effects.

The present invention provides a compound having a Formula I below:

[chemical structure]

wherein: A is CH or N; X is CH or N; R1 is selected from: —SO₂CH₃, —SO₂N(CH₃)₂, —C(O)N(R3)₂, —C(O)R4, and —NHSO₂CH₃; R2 is selected from: —C₃₋₄ alkyl, —OCH(CH₃)₂, and —SCH(CH₃)₂; each R3 is independently selected from: H and —CH₃; R4 is selected from: 4-morpholinyl, 1-piperidinyl, 4-thiomorpholinyl, —NH(CH₂)₃OH, and 4-methyl-1-piperazinyl; and provided that when one of A or X is N the other one of A or X is CH; or pharmaceutically acceptable salts thereof.

The present invention also provides of compounds of Formula I above, or pharmaceutically acceptable salts thereof, wherein both A and X are CH. In another form A is N and X is CH. In still yet another form A is CH and X is N.

The present invention also provides compounds of Formula I, or pharmaceutically acceptable salts thereof, wherein R1 is selected from —C(O)N(R3)₂ or —C(O)R4. In other embodiments when R1 is selected from —C(O)N(R3)₂ or —C(O)R4; R2 is selected from: —C₃₋₄ alkyl —OCH(CH₃)₂, and —SCH(CH₃)₂; more preferably R2 is isopropyl, tert-butyl and —SCH(CH₃)₂. In another form, when R1 is selected from —C(O)N(R3)₂ or —C(O)R4, R2 is selected from: isopropyl, tert-butyl —OCH(CH₃)₂, and —SCH(CH₃)₂; each R3 is independently H, or —CH₃, and R4 is selected from: 4-morpholinyl, 1-piperidinyl, 4-thiomorpholinyl and 4-methyl-1-piperazinyl. In another form R1 is —C(O)N(R3)₂, R2 is selected from: isopropyl, tert-butyl —OCH(CH₃)₂, and —SCH(CH₃)₂; and R3 is independently H, or —CH₃. In another form, R1 is —C(O)R4; R2 is selected from: —C₃₋₄ alkyl, —OCH(CH₃)₂, and —SCH(CH₃)₂; and R4 is selected from 4-morpholinyl, 1-piperidinyl, 4-thiomorpholinyl, and 4-methyl-1-piperazinyl. More preferably R1 is —C(O)R4; R2 is selected from: isopropyl, tert-butyl and —SCH(CH₃)₂; and R4 is 4-morpholinyl, 1-piperidinyl, 4-thiomorpholinyl, and 4-methyl-1-piperazinyl, More preferably R1 is —C(O)R4; R2 is selected from: isopropyl, tert-butyl and —SCH(CH₃)₂; and R4 is 4-morpholinyl or 4-methyl-1-piperazinyl. Still yet more preferably, R1 is —C(O)R4; R2 is tert-butyl; and R4 is 4-methyl-1-piperazinyl.

The present invention also provides compounds of Formula I, or pharmaceutically acceptable salts thereof, wherein, R2 preferably is selected from: —C₃₋₄ alkyl, and —SCH(CH₃)₂. More preferably R2 is selected from: isopropyl, tert-butyl, and —SCH(CH₃)₂. Still more preferably the R2 is isopropyl or tert-butyl.

In one embodiment the present invention also provides compounds of Formula I, or a pharmaceutically acceptable salt thereof, wherein each R3 is —CH₃. In another embodiment the present invention provides compounds of Formula I, or a pharmaceutically acceptable salt thereof, wherein each R3 is H.

The present invention provides compounds of Formula I, or pharmaceutically acceptable salts thereof, wherein R4 is selected from: 4-morpholinyl, 1-piperidinyl, 4-thiomorpholinyl, and 4-methyl-1-piperazinyl or pharmaceutically acceptable salts thereof. More preferably, R4 is selected from: 1-piperidinyl, 4-morpholinyl, and 4-methyl-1-piperazinyl. More preferably R4 is 4-morpholinyl or 4-methyl-1-piperazinyl. Still more preferably R4 is 4-methyl-1-piperazinyl.

The present invention also provides compounds of Formula I, or pharmaceutically acceptable salts thereof, wherein A is CH; R1 is selected from: —SO₂CH₃, —SO₂N(CH₃)₂, —C(O)N(R3)₂, —C(O)R4, and —NHSO₂CH₃; R2 is selected from: —C₃₋₄ alkyl, —OCH(CH₃)₂, and —SCH(CH₃)₂; each R3 is independently H or —CH₃; and R4 is selected from: 4-morpholinyl, 1-piperidinyl, 4-thiomorpholinyl, —NH(CH₂)₃OH and 4-methyl-1-piperazinyl.

The present invention also provides compounds of Formula I, or pharmaceutically acceptable salts thereof, wherein A is CH; X is CH; R1 is —C(O)N(R3)₂, or —C(O)R4; R2 is selected from: —C₃₋₄ alkyl, —OCH(CH₃)₂, and —SCH(CH₃)₂; each R3 is independently H or —CH₃; and R4 is selected from: 4-morpholinyl, 1-piperidinyl, 4-thiomorpholinyl, —NH(CH₂)₃OH and 4-methyl-1-piperazinyl; or pharmaceutically acceptable salts thereof.

The present invention also provides compounds of Formula I, or pharmaceutically acceptable salts thereof, wherein A is CH; X is CH; R1 is —C(O)N(R3)₂, or —C(O)R4; R2 is selected from: —C₃₋₄ alkyl; each R3 is independently H or —CH₃; and R4 is selected from: 4-morpholinyl, 1-piperidinyl, 4-thiomorpholinyl, —NH(CH₂)₃OH and 4-methyl-1-piperazinyl.

The present invention also provides compounds of Formula I, or pharmaceutically acceptable salts thereof, wherein A is CH; X is N; R1 is —C(O)N(R3)₂ and R2 is —C₃₋₄ alkyl; R3 is H or —CH₃. Preferably R2 is tert-butyl.

The present invention also provides compounds of Formula I, or pharmaceutically acceptable salts thereof, wherein A is N; X is CH; R1 is —SO₂CH₃ or —C(O)N(CH₃)₂; and R2 is —C₃₋₄ alkyl. Preferably R2 is tert-butyl.

A particularly preferred compound of the present invention is 4-[5-(3,5-Di-tert-butylphenyl)-1-[4-(4-methylpiperazine-1-carbonyl)phenyl]pyrazol-3-yl]benzoic acid, or a pharmaceutically acceptable salt thereof.

The present invention also provides a pharmaceutical composition that comprises a compound in any of the forms described above for Formula I, or a pharmaceutically acceptable salt thereof, and at least one pharmaceutically acceptable carrier, excipient, or diluent.

The present invention also provides a pharmaceutical composition that comprises a compound in any of the forms described above for Formula I, or a pharmaceutically acceptable salt thereof, least one pharmaceutically acceptable carrier, excipient, or diluent and one or more therapeutic agents.

The present invention provides a method of treating osteoarthritis in a patient in need of treatment. The method comprises administering an effective amount of a compound, in any of the forms described above for Formula I, or a pharmaceutically acceptable salt thereof to the patient.

The present invention also provides a method of treating osteoarthritis in a patient in need of treatment. The method comprises administering an effective amount of a pharmaceutical composition comprising a compound in any of the forms described above for Formula I, or a pharmaceutically acceptable salt thereof to the patient.

The present invention also provides a compound in any of the forms described above for Formula I or a pharmaceutically acceptable salt thereof for use in therapy.

The present invention also provides a compound in any of the forms described above for Formula I or a pharmaceutically acceptable salt thereof for use in the treatment of osteoarthritis, more particularly for the treatment of pain associated with osteoarthritis.

The present invention also provides a compound in any of the forms described above for Formula I or a pharmaceutically acceptable salt thereof for use in the manufacture of a medicament. Preferably the medicament is for treating osteoarthritis. Still more preferably the medicament is for treating the pain associated with osteoarthritis.

The present invention also provides an intermediate according to Formula II

[chemical structure]

wherein: R is selected from C₁₋₄ alkyl, C₁₋₄ haloalkyl, C₃₋₆ cycloalkyl, C₁₋₄ alkyl-C₃₋₆ cycloalkyl, phenyl, and C₁₋₅ alkylphenyl; A is CH or N; X is CH or N R1 is selected from: —SO₂CH₃, —SO₂N(CH₃)₂, —C(O)N(R3)₂, —C(O)R4, and —NHSO₂CH₃; R2 is selected from: —C₃₋₄ alkyl, —OCH(CH₃)₂, and —SCH(CH₃)₂; each R3 is independently selected from: H and —CH₃; R4 is selected from: 4-morpholinyl, 1-piperidinyl, 4-thiomorpholinyl, —NH(CH₂)₃OH, and 4-methyl-1-piperazinyl; and provided that when one of A or X is N, the other one of A or X is CH.

The present invention also provides a process of preparing a compound of Formula I,

[chemical structure]

A is CH or N; X is CH or N; R1 is selected from: —SO₂CH₃, —SO₂N(CH₃)₂, —C(O)N(R4)₂, —C(O)R4, and —NHSO₂CH₃; R2 is selected from: —C₃₋₄ alkyl, —OCH(CH₃)₂, and —SCH(CH₃)₂; each R3 is independently selected from: H, and —CH₃; R4 is selected from: 4-morpholinyl, 1-piperidinyl, 4-thiomorpholinyl, —NH(CH₂)₃OH, and 4-methyl-1-piperazinyl. The process comprising de-esterifying a compound of Formula II;

[chemical structure]

wherein R1-R4 are as described above and R is selected from: C₁₋₄ alkyl, C₁₋₄ haloalkyl, C₃₋₆ cycloalkyl, C₁₋₄ alkyl-C₃₋₆ cycloalkyl, phenyl, and C₁₋₅ alkylphenyl to provide a compound of formula I, or a pharmaceutically acceptable salt thereof.

The present invention also provides a compound which is 4-[5-(3,5-Di-tert-butylphenyl)-1-[4-(4-methylpiperazine-1-carbonyl)phenyl]pyrazol-3-yl]benzoic acid in crystalline form characterized by an X-ray powder diffraction pattern obtained from a CuKα source (λ=1.54056 Å) which comprises peaks at: a) 5.4, 7.5, 14.6, and 19.9+/−0.2 in 2θ; or b) 5.4, 7.5, 14.6, 16.0, 19.4, and 19.9+/−0.2 in 2θ; or c) 5.4, 7.5, 14.6, 15.7, 16.0, 19.4, 19.9 and 22.1+/−0.2 in 2θ.

FIG. 1 is a spectrogram of a representative XRD pattern for crystalline 4-[5-(3,5-Di-tert-butylphenyl)-1-[4-(4-methylpiperazine-1-carbonyl)phenyl]pyrazol-3-yl]benzoic acid. The XRD spectrogram was obtained as described below.

The term alkyl as used herein refers to a carbon substituent which can be a straight chain, e.g., —CH₂CH₂CH₃, —CH₂CH₂CH₂CH₃ or a branched chain, i.e., —CH(CH₃)₂, —C(CH₃)₃ or —CH₂CH(CH₃)₂.

Preferably for all the forms of the compounds described above, the alkyl chain for the R2 substituent group is a branched alkyl chain, preferably an isopropyl alkyl group or a tert-butyl group.

The term C₁₋₄ haloalkyl as used herein refers to a hydrocarbon substituent of one to four carbons where one or more of the hydrogens is replaced with a halogen. The haloalkyl can be a perhalo alkyl where all the hydrogen atoms are replaced with a halogen atom. Alternatively 1, 2, 3, or more hydrogens, can be replaced by a halogen. Further the halogens need not be attached to the same carbon atom.

A “patient” refers to a mammal, preferably a human.

The phrase “pharmaceutically-acceptable salt” refers to salts of the compounds of the invention considered to be acceptable for clinical and/or veterinary use. Pharmaceutically acceptable salts and common methodology for preparing them are well known in the art. See, e.g., P. Stahl, et al., Handbook of Pharmaceutical Salts: Properties, Selection and Use, (VCHA/Wiley-VCH, 2002); S. M. Berge, et al., “Pharmaceutical Salts,” Journal of Pharmaceutical Sciences, Vol. 66, No. 1, January 1977.

The terms and abbreviations used in the instant Schemes, Preparations, Examples and Procedures have their normal meanings unless otherwise designated.

As used herein, the following terms have the meanings indicated: “AcOH” refers to acetic acid, “ATRA” refers to all-trans retinoic acid; “BOP” refers to benzotriazol-1-yloxy)tris(dimethylamino)phosphonium hexafluorophosphate; “CDI” refers to 1,1′-carbonyldiimidazole; “CHAPS” refers to 3-[(3-cholamidopropyl)dimethylammonio]-1-propanesulfonate hydrate; “DCM” refers to dichloromethane; “DDQ” refers to 2,3-dichloro-5,6-dicyano-1,4-benzoquinone or 4,5-dichloro-3,6-dioxo-cyclohexa-1,4-diene-1,2-dicarbonitrile; “DMF” refers to dimethylformamide; “DMSO” refers to methyl sulfoxide; “DTT” refers to dithiothreitol; “EDCI” refers to 1-(3-dimethylaminopropyl)-3-ethylcarbodiimide hydrochloride; “EtOAc” refers to ethyl acetate; “EtOH” refers to ethanol; “FBS” refers to fetal bovine serum; “HEPES” refers to 4-(2-hydroxyethyl)-1-piperazineethanesulfonic acid; “HOBT” refers to 1-hydroxybenzotriazole hydrate; LC-ES/MS refers to liquid chromatography electrospray mass spectroscopy; “MeOH” refers to methanol; “MTBE” refers to methyl t-butyl ether; “PCPNiCl” refers to the reagent wherein the phosphorous-carbon-phosphorous atoms are bound to the nickel in a pincer complex; “SPA” refers to scintillation proximity assay; “TFA” refers to trifluoroacetic acid; “THF” refers to tetrahydrofuran; and “TTNPB” refers to tetrahydro-5,5,8,8-tetramethyl-2-naphthalenyl)-1-propenyl]benzoic acid.

The compounds of the present invention may be prepared by a variety of procedures known in the art as well as the general procedures illustrated in Schemes 1-5 below. However, the following discussion is not intended to be limiting to the scope of the present invention in any way. For example, the specific synthetic steps for each of the routes described may be combined in different ways, or in conjunction with steps from different schemes, to prepare additional compounds of the present invention.

The reagents and starting materials are readily available to one of ordinary skill in the art or may be made by procedures which are selected from standard techniques of organic and heterocyclic chemistry, and the procedures described in the Examples below.

The substituents R1, R2, R3, A, and X are defined as previously indicated. Other variables are defined in the text accompanying the Schemes. Unless specified to the contrary, the naming of the following Preparations and Examples is done using the IUPAC naming feature in Symyx Draw® version 3.2 (Symyx Solutions, Inc.).

[chemical structure]

Scheme 1 illustrates the synthesis of compounds of the invention as shown by formula (8).

In Step A, an aldehyde of formula (I) (A=CH or N) is condensed with 4-acetylbenzoic acid (2) to provide a propenyl benzoic acid of formula (3). The reaction proceeds in a mixture of EtOH and water at a temperature of 10 to 80° C. for 12 h to 2 days.

In Step B, propenyl benzoic acid (3) is esterified to a benzoate (4) using acid catalysis; preferably the benzoate is methyl benzoate prepared using methanesulfonic acid in MeOH at −10 to 50° C. for 4 to 24 h.

In Step C, benzoate (4) is reacted with a phenyl or pyridyl hydrazine of formula (5) (X═CH or N) to provide a dihydropyrazole of formula (6). Preferred conditions use a mixture of 1-butanol and acetic acid at a temperature of 70° C. to the reflux temperature of the solvent for 8 to 24 h.

In Step D, dihydropyrazole (6) is oxidized to a pyrazole benzoate of formula (7). The literature provides a variety of options to the skilled artisan for such an oxidation. Preferred conditions make use of manganese (IV) oxide in a mixture of 1,2-dichloroethane and acetic acid at 50° C. to the reflux temperature of the solvent for 4 to 24 h. Other preferred conditions use DDQ in refluxing toluene.

In Step E, pyrazole benzoate (7) is hydrolyzed to a pyrazole benzoic acid of formula (8) using an inorganic base, preferably lithium hydroxide in a mixture of THF/MeOH or THF/MeOH/water for 4 to 24 h at 0 to 60° C.

The benzaldehydes or pyridine-4-carboxaldehydes of formula (I) are commercially available or can be readily prepared by literature procedures. Likewise the phenyl and pyridyl hydrazines of formula (5) are commercially available or can be readily prepared.

[chemical structure]

Scheme 2 illustrates an alternate means for making compounds of the invention (12) where R2a=I or R2, and R3a is N(R3)₂ or R4.

In Step A, a benzoic acid or a picolinic acid (9) is amidated to form a benzamide or pyridinecarboxamide of formula (10). There are a variety of coupling reagents and reaction conditions available to the skilled artisan for making an amide from a carboxylic acid. Preferred conditions use BOP as a coupling reagent, in an inert solvent, such as DMF, with an organic base, such as diisopropylethylamine in the presence of the appropriate amine Other preferred conditions use EDCI and HOBT in dichloromethane. Alternately, the carbonylimididazole is made in situ using CDI and then reacted with the amine.

In Step B, when R2a=I and A=CH, the iodo t-butylphenyl (10) is transformed to the isopropylthiophenyl of formula (11). The reaction is performed in an inert solvent, such as DMF, using 1,2-diisopropylsulfane, in the presence of zinc and a nickel PCP pincer complex, such as [NiCl{C₆H₃-2,6-(OPPh₂)₂}] (Tetrahedron Lett. 2006, 49, 5059). The reaction proceeds at a temperature of 80-120° C. for 4-24 h.

In Step C, the benzoate of formula (11) or (10) is hydrolyzed as previously described for Scheme 1, Step E.

The benzoic acid or picolinic acid (9) can be made by cyclizing the corresponding hydrazine with a methyl propenoyl benzoate (3) as described for Scheme 1, Step C.

[chemical structure]

Scheme 3 illustrates further chemical modifications leading to compounds of the invention (15).

The nitrophenyl or 2-nitropyridyl (13) can be made by cyclizing the corresponding hydrazine with a propenoyl benzoate (4) as described for Scheme 1, Step C. 4-(Nitrophenyl)hydrazine and 5-hydrazinyl-2-nitro-pyridine are commercially available or can be made using chemistry known in the art. Alternatively, the aniline or 2-aminopyridine (14) can be obtained using other phenylhydrazine or hydrazinopyridine intermediates which are then transformed to the free amine by the skilled artisan. If necessary, appropriate protecting groups can be used.

In Step A, a nitrophenyl or 2-nitropyridyl of formula (13) is reduced to the aniline or 2-aminopyridyl of formula (14). The reduction is performed in a solvent mixture of MeOH and water in the presence of iron and ammonium chloride. The reaction is heated at reflux temperature for 1-8 h.

Following the reduction, the resulting amine is sulfonylated in Step B using methanesulfonyl chloride in the presence of pyridine. Hydrolysis, Step C, is as previously described in Scheme 1, Step E.

[chemical structure]

Scheme 4 illustrates an alternate route to constructing the pyrazole core, leading to compounds of the invention (8).

In Step A the phenyl or pyridyl hydrazine (5) is cyclized with 4-(2-methoxycarbonyl-acetyl)-benzoate (16a, Ra═CO₂Me) or with a 4-(2-cyanoacetyl)benzoate (16b, R═CN) to provide the hydroxypyrazole (17a, Y═OH) (Synlett 2004, 795) or aminopyrazole (17b, Y═NH₂) respectively. The reaction proceeds in a protic solvent, such as MeOH (for the methyl benzoate), at the refluxing temperature of the solvent.

In Step B, the hydroxypyrazole (17a) and the aminopyrazole (17b) are transformed to the bromopyrazole (18a) and the iodopyrazole (18b), respectively. The bromopyrazole (18a) is formed using phosphorous tribromide in an inert solvent such as acetonitrile, at the refluxing temperature of the solvent. The iodopyrazole (18b) is formed by oxidative deamination of the aminopyrazole (17b) using an alkyl nitrite, such as isoamyl nitrite or t-butyl nitrite, in the presence of a suitable iodide source such as copper(I) iodide with or without the addition of diiodomethane. The reaction takes place in an inert solvent, such as acetonitrile, at 60-85° C. over 1 to 12 h.

In Step C, a tert-butylphenyl or tert-pyridyl pyrazole of formula (7) is obtained using a cross-coupling reaction between the bromo or iodopyrazole (18a or 18b) and a phenyl or 4-pyridyl boronate ester (19). Although the boronate ester is shown, it will be known to one skilled in the art that the boronic acids can work equally as well in Suzuki reactions such as these. Furthermore, it is known to the skilled artisan that there are various reaction conditions and Pd catalysts that can be used in such a reaction. The preferred conditions when Z═Br (18a) use a palladium catalyst, such as tetrakis(triphenylphosphine)palladium(0), in an inert solvent such as THF, in the presence of an inorganic base such as aqueous sodium carbonate. The reaction proceeds over 2-24 h at about 50 to 65° C. Preferred conditions when Z═I (18b) make use of bis(triphenylphosphine)palladium(II) chloride in a solvent mixture of THF/water in the presence of a inorganic base, such as potassium carbonate. The reaction proceeds over 2-24 h at about 60° C. to the reflux temperature of the solvent.

Hydrolysis, Step D, is as previously described in Scheme 1, Step E.

The boronate esters (19) or analogous boronic acids can be readily made using literature procedures or by adapting literature procedures (see for example Org Syn 2005, 82, 126).

[chemical structure]

Scheme 5 illustrates another synthetic route to making compounds of the invention (8) where R3a is N(R3)₂ or R4.

In Step A, the aminopyrazole benzoic acid or picolinic acid (20) is acylated to form a benzamide or pyridinecarboxamide of formula (21). There are a variety of coupling reagents and reaction conditions available to the skilled artisan for making an amide from a carboxylic acid. Preferred conditions use CDI, in an inert solvent such as THF, to make the carbonylimidazole in situ. This is followed by reaction with a cyclic amine, such as, morpholine, thiomorpholine, piperidine, or 1-methylpiperazine at 45 to 70° C.

In Step B, the aminopyrazole (21) is converted to the iodopyrazole (22) using a Sandmeyer reaction, as previously described for Scheme 4, Step B.

In Step C, the cross-coupling reaction between the iodopyrazole (22) and the phenyl or 4-pyridyl boronate ester (or boronic acid) proceeds essentially as previously described in Scheme 4, Step C, which is followed by hydrolysis in Step D.

### PREPARATION 1

### 3,5-Di-tert-butylbenzaldehyde

Dissolve 1-bromo-3,5-di-tert-butylbenzene (5.00 g, 18.57 mmol) in THF (50 mL) under a nitrogen atmosphere. Cool to −78° C. Slowly add n-butyllithium (2.5 M in hexanes) (22.29 mL, 55.72 mmol) at −78° C. Stir at −78° C. for about 30 min. Add DMF (4.31 mL, 55.72 mmol) dropwise. Warm the mixture to 0° C. and stir for 2.5 h. Pour aqueous NH₄Cl (30 mL) into the mixture. Extract with EtOAc (3×20 mL). Dry the combined organic portions over Na₂SO₄; filter; collect the filtrate; and concentrate under reduced pressure. Purify the residue using flash chromatography eluting with a gradient of 0-10% EtOAc/petroleum ether to afford the title compound (2.96 g, 73%) as a white solid. LC-ES/MS m/z 219 [M+H]⁺.

### PREPARATION 2

### 1-(Bromomethyl)-3-tert-butyl-5-iodo-benzene

Dissolve 1-tert-butyl-3-iodo-5-methylbenzene (1.14 g, 4.14 mmol) in carbon tetrachloride (20 mL). Add benzoyl peroxide (0.04 g, 0.166 mmol). Heat the mixture to reflux, and add N-bromosuccinimide (1.47 g, 8.28 mmol). Stir the mixture overnight at reflux temperature. Pour the reaction into water (100 mL) and extract with dichloromethane (2×50 mL). Wash the combined organics with saturated NaHCO₃ (50 mL), dry over Na₂SO₄; filter; collect the filtrate; and concentrate under reduced pressure. Purify the crude product by flash chromatography to afford 1-(bromomethyl)-3-tert-butyl-5-iodo-benzene as a mixture with 1-tert-butyl-3-(dibromomethyl)-5-iodobenzene (1.24 g) (about 1.5/1 ratio of mono-bromo/dibromo. ¹H NMR (300 MHz, CDCl₃) δ 1.29-1.30 (m, 9H), 1.31-1.32 (m, 6H), 4.39 (s, 2H), 6.53 (s, 0.43H), 7.34 (s, 1H), 7.48 (s, 0.43H), 7.56 (s, 1H), 7.62-7.65 (m, 1.49H), 7.74 (s, 0.56H).

### PREPARATION 3

### 3-tert-Butyl-5-iodo-benzaldehyde

Dissolve 1-(bromomethyl)-3-tert-butyl-5-iodo-benzene (3.80 g, 10.76 mmol) in dimethyl sulfoxide (20 mL). Heat to 100° C. and stir 4 h. Cool the reaction to room temperature. Partition the mixture between water (40 mL) and EtOAc (40 mL). Dry the organic portion over Na₂SO₄; filter; collect the filtrate; and concentrate under reduced pressure. Purify by flash chromatography (Biotage® system, 80 g cartridge) with a gradient of 0-5% EtOAc/petroleum ether to afford the title compound (1.90 g, 61%). 1H NMR (300 MHz, CDCl₃) δ 1.36 (s, 9H), 7.87 (s, 1H), 7.97 (s, 1H), 8.03 (s, 1H), 9.92 (s, 1H).

### PREPARATION 4

### 3-Bromo-5-tert-butylphenol

Under a nitrogen atmosphere dissolve 1,3-dibromo-5-tert-butylbenzene (10.00 g, 34.25 mmol) in THF (30 mL). Cool to −78° C. Slowly add n-butyllithium (2.5 M in hexanes) (14.38 mL, 35.96 mmol) at −78° C. Stir the resulting mixture for 30 min at −78° C. Add trimethoxyborane (4.88 mL, 42.81 mmol) over 10 min. Warm to room temperature and stir for 1 h. Cool the mixture to 0° C. Add AcOH (13.74 mL, 239.72 mmol) and stir for 10 min. Slowly add hydrogen peroxide (4.11 mL, 134.93 mmol) and water (0.718 mL) and stir 3 h. Add water (5 mL) and extract with EtOAc. Wash the combined organic portions with brine. Purify the crude material by flash chromatography, eluting with petroleum ether/EtOAc (10:1) to afford the title compound (6.46 g, 82%). LC-ES/MS m/z (⁷⁹Br/⁸¹Br) 227/229 [M−H]⁻.

### PREPARATION 5

### 1-Bromo-3-tert-butyl-5-isopropoxy-benzene

Dissolve 3-bromo-5-tert-butylphenol (2.00 g, 8.73 mmol) and 2-bromopropane (1.27 mL, 13.09 mmol) in DMF (10 mL). Add potassium carbonate (3.62 g, 26.19 mmol). Heat to 50° C. and stir 2 h. Dilute with EtOAc (100 mL) and wash the reaction mixture with water (3×20 mL). Dry and concentrate the organic portion under reduced pressure. Purify the crude mixture by flash chromatography, eluting with petroleum ether to afford the title compound (2.00 g, 85%) as a clear liquid. ¹H NMR (CDCl₃ 300 MHz) δ 1.28 (s, 9H), 1.32-1.36 (d, 6H), 4.47-4.52 (m, 1H), 6.81-6.86 (m, 2H), 7.06-7.08 (t, 1H).

### PREPARATION 6

### 3-tert-Butyl-5-isopropoxy-benzaldehyde

Dissolve 1-bromo-3-tert-butyl-5-isopropoxybenzene (2.00 g, 7.38 mmol) in THF (50 mL) under an atmosphere of nitrogen. Cool the solution to −78° C. Add n-butyllithium (2.5 M in hexanes) (8.85 mL, 22.12 mmol) at −78° C. slowly to keep the temperature below −70° C. Stir the mixture for 30 min at −78° C. Add DMF (1.71 mL, 22.12 mmol) dropwise into the mixture at −78° C. Warm the mixture to 0° C. and stir 2.5 h. Quench the reaction with aqueous NH₄Cl. Extract with EtOAc and dry the combined organics over Na₂SO₄; filter; and concentrate under reduced pressure. Purify the resulting residue by flash chromatography on silica, eluting with a gradient of about 0-10% EtOAc/petroleum ether to afford the title compound (1.35 g, 83%). ¹H NMR (CDCl₃, 300 MHz) δ 1.34 (s, 9H), 1.35-1.37 (t, 6H), 4.59-4.67 (m, 1H), 7.18-7.21 (m, 2H), 7.45-7.46 (t, 1H), 9.95 (s, 1H).

### PREPARATION 7

### 4-[(E)-3-(3,5-di-tert-Butylphenyl)prop-2-enoyl]benzoic Acid

Dissolve 4-acetylbenzoic acid (15.00 g, 91.37 mmol) in ethanol (80 mL), and water (40 mL). Add sodium hydroxide (3.65 g, 91.26 mmol). Stir the mixture at room temperature for 30 min. Add 3,5-di-tert-butylbenzaldehyde (20.00 g, 91.60 mmol). Stir the mixture at room temperature for 2 days. Quench the reaction with 2 N HCl (10 mL). Adjust to about pH=2 with 2 N HCl (20 mL). Filter the resulting white solid, washing with ethanol (100 mL). Dry the solid under reduced pressure to afford the title compound (18.30 g, 55%) as a white solid. LC-ES/MS m/z 365 [M+H]⁺.

Prepare the intermediates in Table 1 below, by essentially following the procedure as described in Preparation 7, using the appropriate benzaldehyde with 4-acetylbenzoic acid and 1.05-1.1 eq of solid NaOH or 5 N NaOH. Filter the solids upon acidification, washing with petroleum ether.

TABLE 1

| | | LC-ES/MS |
|---|---|---|
| Prep | Structure and Chemical Name | m/z or NMR |
| 8 | 4-[(E)-3-(3-tert-Butyl-5-iodo-phenyl)prop-2- | 435 |
| | enoyl]benzoic acid | [M + H]⁺ |
| 9* | 4-[(E)-3-(3-tert-Butyl-5-isopropoxy-phenyl)prop- | ¹H NMR (300 |
| | 2-enoyl]benzoic acid | HMz, CDCl₃,) |
| | | consistent |

*5/1 ratio of EtOH/water.

### PREPARATION 10

### Methyl-4-[(E)-3-(3,5-di-tert-butylphenyl)prop-2-enoyl]benzoate

Dissolve 4-[(E)-3-(3,5-di-tert-butylphenyl)prop-2-enoyl]benzoic acid (2.30 g, 6.31 mmol) in methanol (250 mL) and cool to 0° C. Add methanesulfonic acid (4.14 mL, 63.10 mmol) at 0° C. Stir the mixture overnight, allowing to warm to room temperature. Concentrate the mixture under reduced pressure. Add EtOAc (100 mL) to the mixture. Wash the organics with aqueous NaHCO₃ (50 mL). Dry the organic layer over Na₂SO₄; filter; collect the filtrate; and concentrate under reduced pressure. Purify the residue by flash chromatography eluting with a gradient of 0-10% EtOAc/petroleum ether to afford the title compound (1.90 g, 80%) as a white solid. LC-ES/MS m/z 379 [M+H]⁺.

Prepare the intermediates in Table 2 below, by essentially following the procedure as described in Preparation 10, using the appropriate propenoylbenzoic acid.

TABLE 2

| | | LC-ES/MS |
|---|---|---|
| Prep | Structure and Chemical Name | m/z or NMR |
| 11 | Methyl 4-[(E)-3-(3-tert-butyl-5-iodo-phenyl)prop- | 449 |
| | 2-enoyl]benzoate | [M + H]⁺ |
| 12 | Methyl 4-[(E)-3-(3-tert-butyl-5-isopropoxy- | ¹H NMR (300 |
| | phenyl)prop-2-enoyl]benzoate | HMz, CDCl₃,) |
| | | consistent |

### PREPARATION 13

### 2-Methylsulfanyl-5-nitro-pyridine

Dissolve 2-chloro-5-nitropyridine (2.20 g, 13.88 mmol) and triethylamine (3.00 mL, 21.52 mmol) in methanol (20 mL). Add sodium methyl mercaptide (1.00 g, 14.27 mmol) in methanol (10 mL) at room temperature and stir for 2 h. Concentrate the reaction solution under reduced pressure. Add 10% aqueous K₂CO₃ to the resulting residue. Extract the mixture with dichloromethane 3 times. Dry the combined organic portions over Na₂SO₄, filter, and concentrate under reduced pressure to afford the title compound (2.3 g, 13.51 mmol, 97%) as a yellow solid. LC-ES/MS m/z 171 [M+H]⁺.

### PREPARATION 14

### 2-Methylsulfonyl-5-nitro-pyridine

Dissolve 2-(methylthio)-5-nitropyridine (2.30 g, 13.51 mmol) in acetone (20 mL). Add 2 N sulfuric acid (25 mL, 50.00 mmol) dropwise. Add KMnO₄ (3.00 g, 18.98 mmol) in water (50 mL) dropwise to the resulting slurry. Stir the mixture at room temperature overnight. Filter the solid. Stir the solid with a warm mixture of EtOH/MeOH (10:1). Filter the resulting heterogeneous mixture through 2 cm of silica to remove the insoluble salt. Concentrate the filtrate to afford the title compound (1.80 g, 66%) as a pale yellow solid. LC-ES/MS m/z 203 [M+H]⁺.

### PREPARATION 15

### 6-Methylsulfonylpyridin-3-amine

Dissolve 2-(methylsulfonyl)-5-nitropyridine (1.80 g, 8.90 mmol) in water (25 mL) and methanol (25 mL). Add iron (1.49 g, 26.68 mmol), and ammonium chloride (2.86 g, 53.47 mmol). Stir for 1 h at reflux temperature. Filter the mixture, washing with EtOAc. Extract the filtrate with EtOAc. Dry the organic portion over MgSO₄; filter; collect the filtrate; and concentrate the filtrate under reduced pressure to afford the title compound (1.40 g, 91%). LC-ES/MS m/z 173 [M+H]⁺.

### PREPARATION 16

### (6-Methylsulfonyl-3-pyridyl)hydrazine Hydrochloride

Dissolve 6-(methylsulfonyl)pyridin-3-amine (0.50 g, 2.90 mmol) in concentrated hydrochloric acid (6 mL). Add sodium nitrite (0.24 g, 3.48 mmol) in water (10 mL) dropwise slowly at −10 to −15° C. Stir the mixture for 2 h at −10 to −15° C. Add tin dichloride (2.20 g, 11.60 mmol) in concentrated hydrochloric acid (15 mL) dropwise at −5° C. Stir the mixture 1 h at −5° C. Filter the resulting yellow solid washing with diethyl ether to afford the title compound (0.270 g, 42%) as a yellow solid. LC-ES/MS m/z 188 [M+H]⁺.

### PREPARATION 17

### 4-Amino-N,N-dimethyl-benzenesulfonamide

Dissolve 4-acetamidobenzene-1-sulfonyl chloride (1.13 g, 4.84 mmol) in THF (20 mL). Add dimethylamine (2 M in THF, 10 mL, 20.00 mmol) slowly with stirring. Stir the mixture overnight. Concentrate the mixture under reduced pressure. Dissolve the residue in EtOAc (50 mL). Wash the organic portion with 2 N NaOH and brine. Dry over Na₂SO₄, filter; collect the filtrate; and concentrate to dryness. Dissolve the resulting oil in ethanol. Add concentrated hydrochloric acid (10 mL, 116.43 mmol). Heat the mixture to reflux and stir 4 h. Concentrate the material under reduced pressure. Dissolve the residue in EtOAc (50 mL) and water (50 mL). Adjust to about pH=10 with 2 N NaOH. Wash the organic layer with brine; dry over Na₂SO₄; filter; collect the filtrate; and concentrate the filtrate to dryness to afford the title compound (0.85 g, 88%). LC-ES/MS m/z 201 [M+H]⁺.

### PREPARATION 18

### 4-Hydrazino-N,N-dimethyl-benzenesulfonamide Hydrochloride

Dissolve 4-amino-N,N-dimethylbenzenesulfonamide (200 mg, 0.999 mmol) in concentrated hydrochloric acid (4 mL). Cool to 0° C. Add sodium nitrite (80 mg, 1.16 mmol) in water (0.4 mL) dropwise at 0° C. Stir the mixture at 0° C. for 1 h. Add a solution of tin dichloride (760 mg, 4.01 mmol) in concentrated HCl (0.8 mL) dropwise to the mixture at 0° C. Stir the mixture at 0° C. for 1 h. Adjust the solution to about pH=10 with 2 N NaOH. Extract the mixture with EtOAc. Concentrate the organic portion under reduced pressure. Add 2 N HCl (5 mL, 10.00 mmol) and stir the mixture for 1 h. Concentrate the solution under reduced pressure to afford the title compound (180 mg). Use the crude product directly in the next step without further purification. LC-ES/MS m/z 216 [M+H]⁺.

### PREPARATION 19

### 4-[3-(3,5-Di-tert-butylphenyl)-5-(4-methoxycarbonylphenyl)-3,4-dihydropyrazol-2-yl]benzoic Acid

Dissolve (E)-methyl 4-(3-(3,5-di-tert-butylphenyl)acryloyl)benzoate (1.00 g, 2.64 mmol), and 4-hydrazinylbenzoic acid (0.64 g, 4.23 mmol) in 1-butanol (100 mL). Add acetic acid (58 mL) and heat to 120° C. for 20 h. Concentrate the mixture under reduced pressure. Wash the solid with MeOH (3×10 mL) to afford the title compound (1.02 g, 75%) as a white solid.

Prepare the intermediates in Table 3 below, by essentially following the procedure as described in Preparation 19, using the appropriate hydrazine (1.6-2 eq) and the appropriate methyl benzoate in a solvent system of 1-butanol/AcOH varying from a ratio of 5/4 to 10/3 except where noted.

TABLE 3

| | | LC-ES/ |
|---|---|---|
| Prep | Structure and Chemical Name | MS m/z |
| 20 | 4-[3-(3-tert-Butyl-5-iodo-phenyl)-5-(4- | 583 |
| | methoxycarbonylphenyl)- | [M + H]⁺ |
| | 3,4-dihydropyrazol-2-yl]benzoic acid | |
| 21* | 4-[3-(3-tert-Butyl-5-isopropoxy-phenyl)-5-(4- | 515 |
| | methoxycarbonylphenyl)-3,4-dihydropyrazol-2- | [M + H]⁺ |
| | yl]benzoic acid | |
| 22** | Methyl 4-[3-(3,5-di-tert-butylphenyl)-2-(6- | 548.5 |
| | methylsulfonyl-3-pyridyl)-3,4-dihydropyrazol- | [M + H]⁺ |
| | 5-yl]benzoate | |
| 23 | Methyl 4-[3-(3,5-di-tert-butylphenyl)-2-[4- | 598 |
| | (dimethylsulfamoyl)phenyl]-3,4-dihydropyrazol- | [M + Na]⁺ |
| | 5-yl]benzoate | |

*Use 1-butanol/AcOH ratio of 1/3. Purify by preparatory TLC eluting with 2:1 petroleum ether/EtOAc.
**Use 4 eq of 5-hydrazinyl-2-(methylsulfonyl)pyridine.

### PREPARATION 24

### 5-Bromopyridine-2-carboxylic Acid

Add 5-bromopicolinonitrile (1 g, 5.46 mmol) to concentrated HCl (13.4 mL, 139.66 mmol) in a round bottomed flask. Heat the mixture to reflux with stirring overnight. Cool the mixture to room temperature. Filter the resulting white solid, washing with water. Dry the solid under reduced pressure to give 5-bromopyridine-2-carboxylic acid (0.707 g, 64%) as a white solid. LC-ES/MS m/z 202 [M+H]⁺.

### PREPARATION 25

### 5-Bromo-N,N-dimethyl-pyridine-2-carboxamide

Add 5-bromopyridine-2-carboxylic acid (0.71 g, 3.50 mmol) to a solution of dimethylamine hydrochloride (0.32 g, 3.92 mmol), EDCI (0.77 g, 4.02 mmol), HOBT (0.35 g, 2.29 mmol), and triethylamine (1.47 mL, 10.55 mmol) in DMF (10 mL). Stir the mixture for 40 h at room temperature. Concentrate the mixture under reduced pressure. Dissolve the residue in dichloromethane (20 mL) and water (5 mL). Wash the mixture with aqueous NaHCO₃ (2×10 mL). Dry the combined organics over Na₂SO₄; filter; collect the filtrate; and concentrate under reduced pressure. Purify the resulting residue by flash chromatography, eluting with a gradient of 0-60% EtOAc/petroleum ether over 20 min, to afford the title compound (0.67 g, 84%). LC-ES/MS m/z (⁷⁹Br/⁸¹Br) 229/231 [M+H]⁺.

### PREPARATION 26

### tert-Butyl N-[[6-(dimethylcarbamoyl)-3-pyridyl]amino]carbamate

Dissolve tert-butyl carbazate (2.18 g, 16.49 mmol), 5-bromo-N,N-dimethyl-pyridine-2-carboxamide (3.44 g, 15.02 mmol), Pd(OAc)₂ (340 mg, 1.50 mmol), sodium t-butoxide (2.05 g, 21.01 mmol]), and 4,5-bis(diphenylphosphino)-9,9-dimethylxanthene (0.89 g, 1.51 mmol), in toluene (50 mL). Purge the reaction vessel 3 times with nitrogen. Heat the mixture to 85° C. and stir for 6 h. Filter the material through diatomaceous earth, washing with EtOAc (60 mL). Concentrate the mixture under reduced pressure. Purify the resulting residue by flash chromatography, eluting with a gradient of 0-15% MeOH/DCM over 30 min to afford the title compound (0.42 g, 10%). LC-ES/MS m/z 281 [M+H]⁺.

### PREPARATION 27

### Methyl 4-[3-(3,5-di-tert-butylphenyl)-2-[6-(dimethylcarbamoyl)-3-pyridyl]-3,4-dihydropyrazol-5-yl]benzoate

Dissolve di-tert-butyl N-[[6-(dimethylcarbamoyl)-3-pyridyl]amino]carbamate (420 mg, 1.50 mmol) in DCM (20 mL). Add TFA (5 mL) in a single portion with stirring. Stir at room temperature for 2 h. Concentrate the mixture under reduced pressure to afford an oil. Dissolve the oil in 1-butanol (20 mL) and AcOH (5 mL). Add methyl 4-[(E)-3-(3,5-ditert-butylphenyl)prop-2-enoyl]benzoate (600 mg, 1.59 mmol) to the reaction mixture. Purge the reaction vessel 3 times with nitrogen. Heat the mixture to 120° C. and stir for 10 h. Concentrate the mixture under reduced pressure. Purify the resulting residue by preparatory TLC, eluting with 1:1 DCM/EtOAc to afford the title compound (85 mg, 11%). LC-ES/MS m/z 541 [M+H]⁺.

### PREPARATION 28

### Methyl 4-[3-(3,5-di-tert-butylphenyl)-2-(4-nitrophenyl)-3,4-dihydropyrazol-5-yl]benzoate

Dissolve methyl 4-[(E)-3-(3,5-di-tert-butylphenyl)prop-2-enoyl]benzoate (200 mg, 0.528 mmol) and (4-nitrophenyl)hydrazine (90 mg, 0.588 mmol) in MeOH (4 mL). Add methanesulfonic acid (0.14 mL, 2.08 mmoles). Heat the solution to 120° C. with microwave irradiation for 30 min. Quench the reaction with aqueous Na₂CO₃ (0.2 mL). Filter the resulting solid, washing the solid with MeOH to afford the title compound (270 mg, quantitative) as a yellow solid. LC-ES/MS m/z 514 [M+H]⁺.

### PREPARATION 29

### 4-[5-(3,5-Di-tert-butylphenyl)-3-(4-methoxycarbonylphenyl)pyrazol-1-yl]benzoic Acid

Dissolve 4-[3-(3,5-di-tert-butylphenyl)-5-(4-methoxycarbonylphenyl)-3,4-dihydropyrazol-2-yl]benzoic acid (1.02 g, 1.99 mmol) in 1,2-dichloroethane (20 mL). Add acetic acid (77 mL) and manganese (IV) oxide (4.84 g, 55.71 mmol). Heat the mixture to 70° C. and stir overnight. Filter the mixture, washing with dichloromethane. Concentrate the mixture under reduced pressure. Purify the crude product using flash chromatography, eluting with 1:1 dichloromethane:petroleum ether to afford the title compound (1.01 g, 99%) as a white solid. LC-ES/MS m/z 511 [M+H]⁺.

### PREPARATION 30

### Methyl 4-[5-(3,5-di-tert-butylphenyl)-1-[4-(piperidine-1-carbonyl)phenyl]pyrazol-3-yl]benzoate

Dissolve piperidine (0.029 g, 0.353 mmoles) in DMF (6 mL). Add 4-[5-(3,5-di-tert-butylphenyl)-3-(4-methoxycarbonylphenyl)pyrazol-1-yl]benzoic acid (0.120 g, 0.235 mmol) and diisopropylethylamine (0.05 mL, 0.282 mmol). Stir the mixture for about 10 min. Add BOP (0.124 g, 0.282 mmol) and stir the mixture for about 3 h at room temperature. Add water (3 mL) and extract with EtOAc (10 mL). Dry the organic layer over Na₂SO₄; filter; collect the filtrate; and concentrate the filtrate to dryness under reduced pressure. Purify the crude product by preparatory TLC, eluting with 4:1 petroleum ether/EtOAc to afford the title compound (0.108 g, 80%). LC-ES/MS m/z 578 [M+H]⁺.

Prepare the intermediates in Table 4 below, by essentially following the procedure as described in Preparation 30, using the appropriate amine. For example, in Preparation 31 use ammonia (2.0 M solution in methanol).

TABLE 4

| | | LC-ES/ |
|---|---|---|
| Prep | Structure and Chemical Name | MS m/z |
| 31 | Methyl 4-[1-(4-carbamoylphenyl)-5-(3,5-di-tert- | 508 |
| | butylphenyl)pyrazol-3-yl]benzoate | [M − H]⁻ |
| 32 | Methyl 4-[5-(3,5-di-tert-butylphenyl)-1-[4- | 538 |
| | (dimethylcarbamoyl)phenyl]pyrazol-3-yl]benzoate | [M + H]⁺ |
| 33 | Methyl 4-[5-(3,5-di-tert-butylphenyl)-1-[4-(3- | 568 |
| | hydroxypropylcarbamoyl)phenyl]pyrazol-3- | [M + H]⁺ |
| | yl]benzoate | |
| 34 | Methyl 4-[5-(3,5-di-tert-butylphenyl)-1-[4- | no data |
| | (thiomorpholine-4-carbonyl)phenyl]pyrazol-3- | |
| | yl]benzoate | |
| 35 | Methyl 4-[5-(3,5-di-tert-butylphenyl)- | 580 |
| | 1-[4-(morpholine-4-carbonyl)phenyl]pyrazol- | [M + H]⁺ |
| | 3-yl]benzoate | |
| 36 | Methyl 4-[5-(3,5-di-tert-butylphenyl)-1-[4-(4- | 593 |
| | methylpiperazine-1-carbonyl)phenyl]pyrazol-3- | [M + H]⁺ |
| | yl]benzoate | |

### PREPARATION 37

### 4-[5-(3-tert-Butyl-5-iodo-phenyl)-3-(4-methoxycarbonylphenyl)pyrazol-1-yl]benzoic Acid

Dissolve 4-[5-(3-tert-butyl-5-iodo-phenyl)-3-(4-methoxycarbonylphenyl)pyrazol-1-yl]benzoic acid (1.34 g, 2.30 mmol) in 1,2-dichloroethane (50 mL). Add AcOH (10 mL) and manganese (IV) oxide (5.60 g, 64.42 mmol). Stir the mixture overnight at room temperature. Filter the mixture, washing with DCM. Concentrate the filtrate under reduced pressure. Purify the crude material by flash chromatography, eluting with a gradient of 3-25% EtOAc/petroleum ether to afford the title compound (0.98 g, 73%). LC-ES/MS m/z 581 [M+H]⁺.

### PREPARATION 38

### Methyl 4-[5-(3-tert-butyl-5-iodo-phenyl)-1-[4-(dimethylcarbamoyl)phenyl]pyrazol-3-yl]benzoate

Dissolve 4-[5-(3-tert-butyl-5-iodo-phenyl)-3-(4-methoxycarbonylphenyl)pyrazol-1-yl]benzoic acid (1.40 g, 2.41 mmol), dimethylamine hydrochloride (0.43 g, 5.31 mmol), EDCI (1.16 g, 6.03 mmol), and HOBT (0.92 g, 6.03 mmol) in DCM (20 mL). Stir at room temperature overnight. Quench the reaction with aqueous NaHCO₃ (10 mL). Extract with DCM (20 mL). Wash the combined organic portion with aqueous NaHCO₃ (2×10 mL), dry over Na₂SO₄, filter, and concentrate under reduced pressure. Purify the resulting residue by flash chromatography on silica (Biotage® system, 40 g cartridge @ 25 mL/min) with a gradient of 0-60% EtOAc/petroleum ether over 40 min to afford the title compound (1.20 g, 82%). LC-ES/MS m/z 608 [M+H]⁺.

### PREPARATION 39

### Methyl-4-[5-(3-tert-butyl-5-iodo-phenyl)-1-[4-(4-methylpiperazine-1-carbonyl)phenyl]pyrazol-3-yl]benzoate

Prepare the title compound, by essentially following the procedure as described in Preparation 38, using 1-methylpiperazine with 4-[5-(3-tert-butyl-5-iodo-phenyl)-3-(4-methoxycarbonylphenyl)pyrazol-1-yl]benzoic acid. LC-ES/MS m/z 663 [M+H]⁺.

### PREPARATION 40

### Methyl 4-[5-(3-tert-butyl-5-isopropylsulfanyl-phenyl)-1-[4-(4-methylpiperazine-1-carbonyl)phenyl]pyrazol-3-yl]benzoate

Dissolve methyl 4-(5-(3-tert-butyl-5-iodophenyl)-1-(4-(dimethylcarbamoyl)phenyl)-1H-pyrazol-3-yl)benzoate (0.28 g, 0.461 mmol) and 1,2-diisopropylsulfane (0.037 mL, 0.232 mmol) in dry DMF (2 mL). Add zinc (0.03 g, 0.454 mmol), and (SP-4-30-[2,6-bis[(dimethylphosphino-κP)oxy]phenyl-κC]chloro-nickel ((PCP)NiCl) (0.01 g, 0.017 mmol) (reagent prepared according to Tetrahedron Lett. 2006, 49, 5059). Purge the reaction vessel 3 times with nitrogen. Heat the mixture at 110° C. with stirring for 4 h. Quench the mixture with water (20 mL) and extract with EtOAc (3×20 mL). Wash the combined extracts with brine (2×10 mL), dry over Na₂SO₄, filter, and concentrate. Purify the crude mixture by flash chromatography on silica (Biotage® system, 20 g cartridge @ 25 mL/min) eluting with a gradient of 0-20% EtOAc/petroleum ether over 30 min to give a mixture of methyl 4-[5-(3-tert-butyl-5-isopropylsulfanyl-phenyl)-1-[4-(dimethylcarbamoyl)phenyl]pyrazol-3-yl]benzoate and starting material (methyl 4-(5-(3-tert-butyl-5-iodophenyl)-1-(4-(dimethylcarbamoyl)phenyl)-1H-pyrazol-3-yl)benzoate) as a white solid (210 mg). Dissolve the mixture (210 mg) in dry DMF (2 mL). Add 1,2-diisopropylsulfane (0.037 mL, 0.232 mmol), zinc (0.03 g, 0.454 mmol), and (PCP)NiCl (0.01 g, 0.017 mmol). Purge the reaction vessel 3 times with nitrogen. Heat the mixture at 110° C. and stir overnight. Quench the mixture with water (20 mL) and extract with EtOAc (3×20 mL). Wash the combined extracts with brine (2×10 mL), dry over Na₂SO₄, filter, and concentrate under reduced pressure. Purify the crude mixture by flash chromatography on silica (Biotage® system, 20 g cartridge @ 25 mL/min) eluting with a gradient of 0-20% EtOAc/petroleum ether over 30 min to afford 190 mg of crude material. Purify the crude product by preparatory HPLC (Spring Column™ C18, 250×250 mm, 10 μm particle, eluting with a gradient of 75-100% acetonitrile with 0.05% TFA in water) to afford the title compound (0.10 g, 39%) as an oil. LC-MS m/z 556 [M+H]⁺.

### PREPARATION 41

### Methyl 4-[5-(3-tert-butyl-5-isopropylsulfanyl-phenyl)-1-[4-(4-methylpiperazine-1-carbonyl)phenyl]pyrazol-3-yl]benzoate

Prepare the title compound, by essentially following the procedure as described in Preparation 40, using diisopropylsulfane and methyl 4-[5-(3-tert-butyl-5-iodo-phenyl)-1-[4-(4-methylpiperazine-1-carbonyl)phenyl]pyrazol-3-yl]benzoate. LC-ES/MS m/z 611 [M+H]⁺.

### PREPARATION 42

### 4-[5-(3-tert-Butyl-5-isopropoxy-phenyl)-3-(4-methoxycarbonylphenyl)pyrazol-1-yl]benzoic Acid

Dissolve 4-[3-(3-tert-butyl-5-isopropoxy-phenyl)-5-(4-methoxycarbonylphenyl)-3,4-dihydropyrazol-2-yl]benzoic acid (0.50 g, 0.971 mmol) in toluene (10 mL). Add DDQ (0.44 g, 1.94 mmol) and heat the mixture to reflux with stirring for 2 h. Concentrate the reaction under reduced pressure. Purify the residue by preparatory TLC, eluting with 30:1 dichloromethane/MeOH to afford the title compound (0.47 g, 94%). LC-ES/MS m/z 513 [M+H]⁺.

Prepare the intermediates in Table 5 below, by essentially following the procedure as described in Preparation 42, using the appropriate dihydropyrazole. Purify the crude products using preparatory TLC, eluting with petroleum ether/EtOAc.

TABLE 5

| | | LC-ES/ |
|---|---|---|
| Prep | Structure and Chemical Name | MS m/z |
| 43 | Methyl 4-[5-(3,5-di-tert-butylphenyl)-1-(6- | 546 |
| | methylsulfonyl-3-pyridyl)pyrazol-3- | [M + H]⁺ |
| | yl]benzoate | |
| 44 | Methyl 4-[5-(3,5-di-tert-butylphenyl)-1-[6- | 539 |
| | (dimethylcarbamoyl)-3-pyridyl]pyrazol-3- | [M + H]⁺ |
| | yl]benzoate | |
| 45 | Methyl 4-[5-(3,5-di-tert-butylphenyl)-1-(4- | 512 |
| | nitrophenyl)pyrazol-3-yl]benzoate | [M + H]⁺ |
| 46 | Methyl 4-[5-(3,5-di-tert-butylphenyl)-1-[4- | 574 |
| | (dimethylsulfamoyl)phenyl]pyrazol-3- | [M + H]⁺ |
| | yl]benzoate | |

### PREPARATION 47

### Methyl 4-[5-(3-tert-butyl-5-isopropoxy-phenyl)-1-[4-(4-methylpiperazine-1-carbonyl)phenyl]pyrazol-3-yl]benzoate

Dissolve 1-methylpiperazine (70 mg, 0.702 mmol) and 4-[5-(3-tert-butyl-5-isopropoxy-phenyl)-3-(4-methoxycarbonylphenyl)pyrazol-1-yl]benzoic acid (180 mg, 0.351 mmol) in dichloromethane (10 mL). Add 1-hydroxybenzotriazole (118 mg, 0.878 mmol) and EDCI (168 mg, 0.878 mmol). Stir the mixture 3 h at room temperature. Add water (3 mL) and aqueous NaHCO₃ (10 mL) and extract with EtOAc (3×10 mL). Dry the combined organics over Na₂SO₄, filter, and concentrate to dryness. Purify the resulting residue by preparatory TLC, eluting with 10:1 DCM/MeOH to afford the title compound (140 mg, 67%). LC-ES/MS m/z 595 [M+H]⁺.

### PREPARATION 48

### Methyl 4-[1-(4-aminophenyl)-5-(3,5-di-tert-butylphenyl)pyrazol-3-yl]benzoate

Dissolve methyl 4-(5-(3,5-di-tert-butylphenyl)-1-(4-nitrophenyl)-1H-pyrazol-3-yl)benzoate (230 mg, 0.450 mmol) in MeOH (8 mL) and water (8 mL). Add iron (80 mg, 1.430 mmol) and ammonium chloride (120 mg, 2.240 mmol) in a single portion. Heat the mixture to reflux and stir 2 h. Filter the mixture, washing with EtOAc. Extract the mixture 3 times with EtOAc. Dry the combined organic portions over Na₂SO₄, filter, and concentrate under reduced pressure to afford the title compound (210 mg, 97%). LC-ES/MS m/z 482 [M+H]⁺.

### PREPARATION 49

### Methyl 4-[5-(3,5-di-tert-butylphenyl)-1-[4-(methanesulfonamido)phenyl]pyrazol-3-yl]benzoate

Dissolve methyl 4-[1-(4-aminophenyl)-5-(3,5-di-tert-butylphenyl)pyrazol-3-yl]benzoate (210 mg, 0.436 mmol) in DCM (10 mL). Add pyridine (0.04 mL, 0.495 mmol) and stir for 5 min. Add methanesulfonyl chloride (0.04 mL, 0.517 mmol) in a single portion and stir overnight. Quench the reaction with aqueous Na₂CO₃. Extract the mixture 3 times with EtOAc. Dry the combined organic portion over Na₂SO₄, filter, and concentrate to dryness under reduced pressure. Purify the resulting residue by flash chromatography (Biotage® system, 20 g cartridge @ 25 mL/min) eluting with a gradient of 8-60% EtOAc/petroleum ether to afford the title compound (230 mg, 94%). LC-ES/MS m/z 560 [M+H]⁺.

### PREPARATION 50

### 2-(3,5-Di-tert-butylphenyl)-4,4,5,5-tetramethyl-1,3,2-dioxaborolane

Bubble nitrogen through DMF (150 mL) for about 15 min prior to adding the reagents. Then dissolve 1-bromo-3,5-di-tert-butylbenzene (20.93 g, 77.74 mmol), bis(pinacolato)diboron (22.70 g, 89.40 mmol), and (1,1′-bis(diphenylphosphino)ferrocene)palladium(II) chloride (3.17 g, 3.89 mmol) in the DMF. Stir for 10 min. Add potassium acetate (22.89 g, 233.23 mmol) and bubble argon through the solution for 7 min. Heat the reaction to 85° C. with stirring for 24 h. Dilute the reaction with water (1.5 L). Collect the resulting brown precipitate by vacuum filtration and washing with water. Dissolve the residue in dichloromethane. Dry this solution over sodium sulfate, filter; collect the filtrate; and concentrate under reduced

pressure. Triturate the resulting solids with hot hexanes (400 mL) and filter, washing with hexanes. Concentrate the filtrate to a volume of approximately 300 mL. Place the solution in a freezer overnight. Collect the solid by vacuum filtration and rinsing with cold hexanes. Concentrate the filtrate under reduced pressure to a volume of 150 mL. Cool this mixture in the freezer for about 1.5 h. Collect the resulting solids by vacuum filtration and rinse with cold hexanes. Combine the 2 crops and dry under high vacuum to afford the title compound (21.85 g, 89%) as a light tan crystalline solid. ¹H NMR (400 MHz, CDCl₃) δ 1.33 (s, 12H), 1.33 (s, 18H), 7.53 (t, J=2.0 Hz, 1H), 7.65 (d, J=2.0 Hz, 2H).

### PREPARATION 50A: ALTERNATE PROCEDURE

### 2-(3,5-Di-tert-butylphenyl)-4,4,5,5-tetramethyl-1,3,2-dioxaborolane

Charge a reactor with 1-bromo-3,5-di-tert-butylbenzene (182.0 g, 1.115 mol), bis (pinacolato)diboron (197.4 g, 1.281 mol), [1,1′-bis(diphenylphosphino)ferrocene]palladium(II) chloride (27.6 g, 0.056 mol), potassium acetate (199.0 g, 3.347 mol) and DMF (1.2 L). Heat the resulting solution to 85° C. for 5 h. Cool the reaction mixture to 25° C. and add water (6 L) to form a brown precipitate. Filter and wash the solid with water. Collect the solid. Dissolve the solid in DCM (1.82 L), dry over Na₂SO₄, filter; collect the filtrate; and evaporate the solvent. Triturate the residue with hot hexane (3.2 L) and filter to remove the catalyst. Concentrate the filtrate to approximately 1.8 L. Cool this solution to 15° C. and stir for 48 h. Filter to collect the solid and dry in the open air to provide the title compound (150.0 g, 70%). LC-ES/MS m/z 317 [M+H]⁺.

### PREPARATION 51

### 3-tert-Butyl-5-(4,4,5,5-tetramethyl-1,3,2-dioxaborolan-2-yl)phenol

Dissolve 3-bromo-5-tert-butylphenol (2.29 g, 10.00 mmol), 4,4,4′,4′,5,5,5′,5′-octamethyl-2,2′-bi-1,3,2-dioxaborolane (3.046 g, 12.0 mmol), (1,1′-bis(diphenylphosphino)ferrocene)palladium(II) chloride (0.820 g, 1.00 mmol), 1,1′-bis(diphenylphosphino)ferrocene (0.560 g, 1.00 mmol), and potassium acetate (2.94 g, 30.00 mmol) in 1,4-dioxane (80 mL). Purge the reaction vessel 3 times with nitrogen. Heat the mixture to 80° C. and stir overnight. Filter the mixture through diatomaceous earth, rinsing the solid cake with EtOAc. Concentrate the filtrate under reduced pressure. Purify the crude mixture by flash chromatography on silica (ISCO® system, 20 g cartridge @ 25 mL/min) eluting with a gradient of 0-20% EtOAc/petroleum ether over 30 min to afford the title compound (2.26 g; 82%). LC-ES/MS m/z 275 [M−H]⁻.

### PREPARATION 52

### 2-(3-tert-Butyl-5-isopropoxy-phenyl)-4,4,5,5-tetramethyl-1,3,2-dioxaborolane

Dissolve 3-tert-butyl-5-(4,4,5,5-tetramethyl-1,3,2-dioxaborolan-2-yl)phenol (200 mg, 0.724 mmol), and 2-bromopropane (178 mg, 1.448 mmol) in DMF (3 mL). Add potassium carbonate (300 mg, 2.149 mmol) in a single portion with stirring. Heat the mixture to 90° C. while stirring overnight. Cool the mixture to room temperature and dilute with EtOAc (50 mL). Wash the combined organics with water and brine; dry over Na₂SO₄; filter; collect the filtrate; and concentrate under reduced pressure. Purify the crude mixture by flash chromatography on silica (ISCO® system, 20 g cartridge @ 30 mL/min) eluting with a gradient of 0-50% EtOAc/petroleum ether over 20 min to afford the title compound (152 mg, 66%). LC-ES/MS m/z 319 [M+H]⁺.

### PREPARATION 53

### 2,6-Di-tert-butyl-4-(4,4,5,5-tetramethyl-1,3,2-dioxaborolan-2-yl)pyridine

Add 1,5-cyclooctadiene)(methoxy)iridium (I) dimer (Ir(OMe)(COD))₂) (0.05 g, 0.075 mmol), 4-tert-butyl-2-(4-tert-butyl-2-pyridyl)pyridine (0.04 g, 0.15 mmol), and bis(pinacolato)diboron (2.67 g, 10.50 mmol) to hexane (30 mL) which has been purged with nitrogen for 20 min. Place in a preheated oil bath at 55° C. Stir for 10 min. Add 2,6-di-tert-butylpyridine (3.81 g, 19.90 mmol) and heat at 55° C. for 72 h. Cool the mixture to room temperature and concentrate under reduced pressure to give the title compound (5.20 g, 82%). LC-ES/MS (m/z) 318 [M+H]⁺.

### PREPARATION 54

### 3-Bromo-5-formyl-tert-butylbenzene

Dissolve 1,3-dibromo-tert-butylbenzene (6.093 g, 20.87 mmol) in 50 mL THF (50 mL) and cool to −78° C. Add n-butyl lithium (2.5 M in hexanes) (9.18 mL, 22.95 mmol) dropwise over 10 min and stir for 15 min. Add DMF (3.23 mL, 41.73 mmol) in one portion and stir for 30 min. Dilute the reaction mixture with EtOAc (40 mL) and 1 N HCl (40 mL). Extract the aqueous layer with EtOAc (2×40 mL). Wash the combined organic portions with brine (100 mL). Dry over sodium sulfate; filter; collect filtrate; and concentrate under reduced pressure. Purify the resulting residue by flash chromatography, eluting with a gradient of hexanes to 10% EtOAc/hexanes over 30 min to give the title compound (4.02 g) as a light yellow oil. ¹H NMR (400 MHz, DMSO-d₆): δ 1.27-1.27 (m, 9H), 7.83 (s, 2H), 7.89-7.88 (m, 1H), 9.93 (s, 1H).

### PREPARATION 55

### 1-(3-Bromo-5-tert-butylphenyl)ethanol

Dissolve 3-bromo-5-formyl-tert-butylbenzene (3.624 g, 15.03 mmol) in diethyl ether (50 mL). Add methylmagnesium bromide (5.51 mL, 16.53 mmol) slowly over 10 min. Stir the reaction mixture for 18 h. Pour the reaction mixture into saturated ammonium chloride (50 mL) and extract with EtOAc (2×50 mL). Dry the organic portions over sodium sulfate, filter, and concentrate under reduced pressure. Purify the resulting residue by flash chromatography, eluting with a gradient of hexanes to 10% EtOAc/hexanes over 30 min to give the title compound (3.49 g) as a clear oil. ¹H NMR (400 MHz, DMSO-d₆) δ 1.23-1.22 (s, 9H), 1.25 (d, J=6.5 Hz, 3H), 4.68-4.62 (m, 1H), 5.19 (d, J=4.4 Hz, 1H), 7.32-7.28 (m, 3H).

### PREPARATION 56

### 1-(3-Bromo-5-tert-butylphenyl)ethanone

Dissolve 1-(3-bromo-5-tert-butylphenyl)ethanol (3.49 g, 13.57 mmol) in chloroform (100 mL) and then add pyridinium chlorochromate (4.48 g, 20.36 mmol). Stir the mixture for 72 h at room temperature. Add 5 N NaOH (150 mL) and stir until the precipitate dissolves. Extract the aqueous layer with dichloromethane (2×100 mL). Wash the combined organic portions with 1 N HCl (150 mL). Dry the organics over sodium sulfate; filter; collect the filtrate; and concentrate under reduced pressure. Purify the residue by flash chromatography eluting with a gradient of hexanes to 20% EtOAc/hexanes over 30 min to give the title compound (2.62 g) as a clear oil. ¹H NMR (400 MHz, DMSO-d₆) δ 1.26 (s, 9H), 2.55 (s, 3H), 7.77 (t, J=1.8 Hz, 1H), 7.85 (t, J=1.6 Hz, 1H), 7.87 (t, J=1.6 Hz, 1H).

### PREPARATION 57

### 2-(3-Bromo-5-tert-butyl-phenyl)propan-2-ol

Dissolve 1-(3-bromo-5-tert-butylphenyl)ethanone (0.80 g, 3.15 mmol) in diethyl ether (40 mL). Add methylmagnesium bromide (1.58 mL, 4.73 mmol) and then stir for 72 h. Pour the reaction into 1 N hydrochloric acid (50 mL). Extract with diethyl ether (2×50 mL). Dry the combined organic portions over sodium sulfate; filter; concentrate the filtrate; and concentrate under reduced pressure to afford the title compound (0.82 g, 96%) as a white solid. ¹H NMR (400 MHz, DMSO-d₆) δ 1.24 (s, 9H), 1.38 (s, 6H), 5.09 (s, 1H), 7.31 (t, J=1.8 Hz, 1H), 7.40 (t, J=1.7 Hz, 1H), 7.44 (t, J=1.6 Hz, 1H).

### PREPARATION 58

### 1-Bromo-3-tert-butyl-5-isopropyl-benzene

Dissolve 2-(3-bromo-5-tert-butyl-phenyl)propan-2-ol (0.82 g, 3.02 mmol) in dichloromethane (20 mL). Add TFA (2.29 mL, 30.24 mmol) and then triethylsilane (2.42 mL, 15.12 mmol). Stir the reaction for 18 h. Pour the reaction into saturated sodium bicarbonate (50 mL) and extract two times with DCM (2×40 mL). Dry the combined organic portions over sodium sulfate; filter; collect the filtrate; and concentrate under reduced pressure. Purify the resulting residue by flash chromatography, eluting with a gradient of hexanes to 10% EtOAc/hexanes over 30 min to give the title compound (0.60 g, 78%) as a clear liquid. ¹H NMR (400 MHz, DMSO-d₆) δ 1.16 (d, J=6.9 Hz, 6H), 1.23 (s, 9H), 2.89-2.82 (m, 1H), 7.22-7.20 (m, 2H), 7.30 (t, J=1.8 Hz, 1H).

### PREPARATION 59

### (3-tert-Butyl-5-isopropyl-phenyl)boronic Acid

Dissolve 1-bromo-3-tert-butyl-5-isopropylbenzene (0.35 g, 1.37 mmol) in THF (10 mL) under a nitrogen atmosphere. Cool the solution to −78° C. Add n-butyllithium (2.5 M in hexanes) (0.66 mL, 1.65 mmol) and stir the mixture at −78° C. for 20 min. Add trimethylborane (0.2 mL, 1.76 mmol) at −78° C. and stir at −78° C. for 2 h. Quench the reaction with water (20 mL). Extract the mixture with EtOAc (2×40 mL). Collect the EtOAc extracts and purify the crude mixture using preparatory TLC, eluting with 1:5 EtOAc/petroleum ether to afford the title compound (0.19 g, 63%). LC-ES/MS m/z 219 [M−H]⁻.

### PREPARATION 60

### Methyl 4-[5-hydroxy-1-(4-methylsulfonylphenyl)pyrazol-3-yl]benzoate

Dissolve 4-methylsulfonylphenylhydrazine hydrochloride (13.40 g, 57.15 mmol), and 4-(2-methoxycarbonyl-acetyl)-benzoic acid methyl ester (10.00 g, 42.3 mmol) in MeOH (150 mL). Heat the mixture to reflux and stir overnight. Cool the reaction to room temperature. Add MeOH (50 mL) and cool to 0° C. Filter the solids using vacuum filtration; then wash the solids with cold MeOH. Dry the solids under reduced pressure to give the title compound (14.3 g, 91%) as a light tan solid. LC-ES/MS m/z 373 [M+H]⁺.

### PREPARATION 61

### Methyl 4-[5-bromo-1-(4-methylsulfonylphenyl)pyrazol-3-yl]benzoate

Dissolve methyl 4-[5-hydroxy-1-(4-methylsulfonylphenyl)pyrazol-3-yl]benzoate (2.00 g, 5.371 mmol) in acetonitrile (8 mL). Add phosphorus tribromide (2.55 mL, 26.85 mmol). Heat the reaction to reflux and stir overnight. Add phosphorus tribromide (1.273 mL, 13.43 mmol) and stir for 72 h. Add phosphorus tribromide (1.27 mL, 13.43 mmol) and stir for 24 h. Cool the reaction to room temperature. Slowly pour the mixture over saturated aqueous sodium bicarbonate. Extract the resulting mixture with DCM. Concentrate the combined extracts under reduced pressure. Purify the resulting residue by flash chromatography over silica gel (40 g) with a gradient of 0-5% EtOAc/DCM to afford the title compound (0.95 g, 41%) as a white crystalline solid. LC-ES/MS m/z (⁷⁹Br/⁸¹Br) 435/437 [M+H]⁺.

### PREPARATION 62

### Methyl 4-[5-(3,5-di-tert-butylphenyl)-1-(4-methylsulfonylphenyl)pyrazol-3-yl]benzoate

Add methyl 4-[5-bromo-1-(4-methylsulfonylphenyl)pyrazol-3-yl]benzoate (75 mg, 0.172 mmol), 2-(3,5-di-tert-butylphenyl)-4,4,5,5-tetramethyl-1,3,2-dioxaborolane (82 mg, 0.258 mmol), (tetrakis(triphenylphosphine)palladium (40 mg, 0.034 mmol), THF (1.7 mL), and 2 N aqueous sodium carbonate (0.284 mL, 0.569 mmol) to an 8 mL screw cap vial (containing septa) equipped with a stir bar. Purge with argon for 1-2 min. Place the reaction flask in a 65° C. oil bath and stir the reaction overnight. Cool the reaction to room temperature and dilute with water. Extract the aqueous with EtOAc. Concentrate the combined extracts under reduced pressure. Purify by radial chromatography (silica gel, 2 mm plate) eluting with 20-50% EtOAc gradient in hexane to afford the title compound (60 mg, 64%) as a white solid foam. LC-ES/MS m/z 545 [M+H]⁺.

### PREPARATION 63

### Methyl 4-[5-amino-1-(4-cyanophenyl)pyrazol-3-yl]benzoate

Suspend of hydrazinylbenzonitrile hydrochloride (1.86 g, 10.97 mmol) and methyl 4-(2-cyanoacetyl)benzoate (2.03 g, 9.99 mmol) in MeOH (40 mL). Heat the reaction to reflux while stirring overnight. Cool the reaction mixture to room temperature. Filter the mixture, washing the solid with petroleum ether (40 mL). Dry the resulting solid in vacuo to afford product (2.02 g). Concentrate the filtrate under reduced pressure. Purify the resulting residue by flash chromatography on silica gel (Biotage® system, 40 g cartridge @ 40 mL/min) with a gradient of 0-70% EtOAc/DCM (40 min) to afford additional product (0.42 g). Combine the two lots of purified product (2.44 g, 77%). LC-ES/MS m/z 319 [M+H]⁺.

### PREPARATION 64

### Methyl 4-[1-(4-cyanophenyl)-5-iodo-pyrazol-3-yl]benzoate

Suspend methyl 4-[5-amino-1-(4-cyanophenyl)pyrazol-3-yl]benzoate (2.02 g, 6.4 mmol) and copper(I) iodide (1.21 g, 6.4 mmol) in acetonitrile (80 mL). Add t-butyl nitrite (1.31 g, 12.7 mmol) while stirring. Heat the mixture to 75° C. while stirring for 3 h. Dilute the mixture with EtOAc (50 mL). Wash the organics with dilute Na₂S₂O₃ (3×) and brine. Dry the organics over Na₂SO₄; filter, collect the filtrate; and concentrate to dryness under reduced pressure. Purify the crude mixture by flash chromatography on silica (ISCO® system, 20 g cartridge @ 25 mL/min) with a gradient of 0-50% EtOAc/petroleum ether over 30 min to afford the title compound (1.54 g, 57%). LC-ES/MS m/z 430 [M+H]⁺.

### PREPARATION 65

### Methyl 4-[1-(4-carbamoylphenyl)-5-iodo-pyrazol-3-yl]benzoate

Dissolve methyl 4-[1-(4-cyanophenyl)-5-iodo-pyrazol-3-yl]benzoate (0.206 g, 0.480 mmol) in TFA (3 mL). Add sulfuric acid (0.75 mL) slowly while stirring the mixture. Heat the mixture to 45° C. with stirring overnight. Pour the mixture into ice water and extract with isopropanol/DCM (1:2 ratio, 3×50 mL). Wash the combined organics with brine; dry over Na₂SO₄; filter; collect the filtrate; and concentrate to dryness under reduced pressure to afford the title compound (0.205 g, 96%). LC-ES/MS m/z 448 [M+H]⁺.

### PREPARATION 66

### 4-[5-Amino-3-(4-methoxycarbonylphenyl)pyrazol-1-yl]benzoic Acid

Suspend 4-hydrazinylbenzoic acid (1.67 g, 10.98 mmol) and methyl 4-(2-cyanoacetyl)benzoate (2.03 g, 9.99 mmol) in methanol (40 mL). Heat to reflux and stir overnight. Cool the reaction mixture to room temperature. Filter the resulting solid, washing with petroleum ether to afford the title compound (2.92 g, 87%). LC-ES/MS m/z 338 [M+H]⁺.

### PREPARATION 66A: ALTERNATE PROCEDURE

### 4-[5-Amino-3-(4-methoxycarbonylphenyl)pyrazol-1-yl]benzoic Acid

Charge a reactor with acetic acid (25 L), 4-hydrazinobenzoic acid hydrochloride (1115.0 g, 5.911 mol) and methyl 4-cyanoacetylbenzoate (1200.0 g, 5.911 mol) at 13° C. Heat the mixture to 80° C. and stir for 20 h. Cool the reaction mixture to 20° C. and filter to give a yellow filter cake. Triturate the filter cake with hexane (5 L) and filter to give the title compound (1621 g, 81%) as a yellow solid. LC-ES/MS m/z 338 [M+H]⁺.

### PREPARATION 67

### Methyl 4-[5-amino-1-[4-(4-methylpiperazine-1-carbonyl)phenyl]pyrazol-3-yl]benzoate

Charge a reactor with THF (18.6 L) and 4-[5-amino-3-(4-methoxycarbonylphenyl)pyrazol-1-yl]benzoic acid (620 g, 1.840 moles) at 15° C. Add CDI (387 g, 2.392 mol) in batches, heat the mixture to reflux, and stir for 2.5 h. Add N-methylpiperizine (360 mL, 2.760 mol) dropwise at reflux temperature over 25 min. Then stir for 17 h at reflux. Cool to 20° C. and add water (7 L) and EtOAc (18 L). Separate the two phases. Extract the aqueous with EtOAc (2×10 L). Combine the organic layers; wash with 1 M NaOH (2.5 L); dry with Na₂SO₄; filter; collect the filtrate; and concentrate under reduced pressure to give a yellow solid. Add a solvent mixture of heptane/MTBE/MeOH (4/4/1, 10 L) and stir for 0.5 h. Collect the solid by filtration. Dry the resulting material to give the title compound (610 g, 77%) as a yellow solid. LC-ES/MS m/z 420 [M+H]⁺.

### PREPARATION 68

### Methyl 4-[5-iodo-1-[4-(4-methylpiperazine-1-carbonyl)phenyl]pyrazol-3-yl]benzoate

Charge a reactor with acetonitrile (21 L), methyl 4-[5-amino-1-[4-(4-methylpiperazine-1-carbonyl)phenyl]pyrazol-3-yl]benzoate (1070 g, 2.554 mol), diiodomethane (1362 g, 5.101 mol) and copper (I) iodide (970 g, 5.105 mol). Heat the mixture to 80° C. with stirring. Add isoamyl nitrite (896 g, 7.658 mol) dropwise at 80° C., and then stir the resultant suspension at 80° C. for 1 h. Cool the mixture to 17° C. and allow to stand for 48 h. Add saturated aqueous solution of NH₄Cl (10 L) and EtOAc (50 L). Separate the two phases. Wash the organic layer with a saturated aqueous solution of Na₂S₂O₃ (10 L), and dry over Na₂SO₄. Filter; collect the filtrate; and evaporate the filtrate to give an orange oil. Purify the residue by column chromatography, eluting with DCM/MeOH (from 50/1 to 5/1) to provide the title compound (381 g, 28%) as a brown solid. LC-ES/MS m/z 531 [M+H]⁺.

### PREPARATION 69

### Methyl 4-[5-iodo-1-[4-(morpholine-4-carbonyl)phenyl]pyrazol-3-yl]benzoate

Suspend 4-(5-amino-3-(4-(methoxycarbonyl)phenyl)-1H-pyrazol-1-yl)benzoic acid (2.56 g, 7.60 mmol) and copper(I) iodide (1.44 g, 7.60 mmol) in acetonitrile (80 mL). Add t-butyl nitrite (1.8 mL, 15.1 mmol) in a single portion. Heat the mixture to 75° C. and stir 3 h. Filter the mixture, washing the solid with acetonitrile to afford a crude mixture of 4-(5-iodo-3-(4-(methoxycarbonyl)phenyl)-1H-pyrazol-1-yl)benzoic acid (3.7 g). Dissolve 4-(5-iodo-3-(4-(methoxycarbonyl)phenyl)-1H-pyrazol-1-yl)benzoic acid (3.7 g) in dry DMF (50 mL). Add diisopropylethylamine (1.8 mL, 10.3 mmol) and morpholine (1.2 mL, 13.8 mmol) and stir 10 min. Add BOP (4.52 g, 10.2 mmol) and stir the mixture 24 h. Quench the mixture with water (200 mL). Extract the mixture with EtOAc (4×60 mL). Wash the combined organic portions with water (4×50 mL) and brine (2×50 mL); dry over Na₂SO₄; filter; collect the filtrate; and concentrate under reduced pressure. Purify the crude product by preparatory HPLC (Waters Sunfire™ column C18, 4.6 mm×150 mm, 5 μm particle size) eluting with a gradient of 45-100% acetonitrile with 0.05% TFA in water to afford the title compound (0.88 g, 23%). LC-ES/MS m/z 518 [M+H]⁺.

### PREPARATION 70

### Methyl 4-[1-[4-(dimethylcarbamoyl)phenyl]-5-iodo-pyrazol-3-yl]benzoate

Dissolve methyl 4-[1-(4-carbamoylphenyl)-5-iodo-pyrazol-3-yl]benzoate (223 mg, 0.499 mmol) in DMF (5 mL). Cool to 0° C. Add sodium hydride (60% in oil) (60 mg, 1.5 mmol) in small portions over 10 min with stirring at 0° C. Stir the mixture 1 h and add methyl iodide (93 μL, 1.5 mmol) in a single portion. Allow the mixture to warm to room temperature and stir 18 h. Quench the reaction with aqueous NH₄Cl (20 mL). Extract the mixture with EtOAc (3×20 mL). Wash the combined organic portions with brine (2×20 mL), dry over Na₂SO₄, filter, and concentrate to dryness. Purify the crude mixture by flash chromatography on silica (Biotage® system, 20 g cartridge @ 25 mL/min) eluting with a gradient of 0-40% EtOAc/dichloromethane to afford the title compound (175 mg, 74%). LC-ES/MS m/z 476 [M+H]⁺.

### PREPARATION 71: ALTERNATE ROUTE TO INTERMEDIATE OF PREPARATION 36

### Methyl 4-[5-(3,5-ditert-butylphenyl)-1-[4-(4-methylpiperazine-1-carbonyl)phenyl]pyrazol-3-yl]benzoate

Charge a reactor with methyl 4-[5-iodo-1-[4-(4-methylpiperazine-1-carbonyl)phenyl]pyrazol-3-yl]benzoate (380 g, 0.720 mol), 2-(3,5-di-tert-butylphenyl)-4,4,5,5-tetramethyl-1,3,2-dioxaborolane (296 g, 0.936 mol), bis(triphenylphosphine) palladium(II) chloride (50 g, 0.072 mol), potassium carbonate (297 g, 2.150 mol), THF (11.4 L) and water (0.76 L). Heat the reaction to 85° C. for 1 h. Cool to 25° C. and filter to give the two-layer filtrate. Separate the phases, and extract the aqueous layer with EtOAc (2×5 L). Combine the organic layers; dry with Na₂SO₄; filter; collect the filtrate; and evaporate the solvent to give a thick, dark brown oil. Purify the oil by column chromatography, eluting with CH₂Cl₂/MeOH (from 50/1 to 20/1) to give the product as a thick brown oil. Triturate the oil in a solvent mixture of acetonitrile/hexane/MTBE (2/1/1, 2 L) for 1 h. Filter to collect the solid and dry in the open air to give the product of the title compound (360 g, 84%) as a gray solid. LC-ES/MS m/z 593 [M+H]⁺.

### PREPARATION 72

### Methyl 4-[5-(3-tert-butyl-5-isopropoxy-phenyl)-1-(4-carbamoylphenyl)pyrazol-3-yl]benzoate

Dissolve methyl 4-[1-(4-carbamoylphenyl)-5-iodo-pyrazol-3-yl]benzoate (116 mg, 0.259 mmol), 2-(3-tert-butyl-5-isopropoxy-phenyl)-4,4,5,5-tetramethyl-1,3,2-dioxaborolane (100 mg, 0.314 mmol), and potassium carbonate (108 mg, 0.781 mmol) in THF (15 mL), and water (3 mL). Add bis(triphenylphosphine)palladium(II) chloride (30 mg, 0.043 mmol). Purge the reaction vessel 3 times with nitrogen. Heat the mixture to 85° C. with stirring for 4 h. Dilute the mixture with EtOAc (50 mL). Wash the combined organics with brine; dry over Na₂SO₄; filter; collect the filtrate; and concentrate to dryness under reduced pressure. Purify the crude mixture by flash chromatography on silica (Biotage® system, 12 g cartridge @ 25 mL/min) eluting with a gradient of 0-50% EtOAc/dichloromethane over 25 min to afford the title compound (105 mg, 79%). LC-ES/MS m/z 512 [M+H]⁺.

Prepare the intermediates in Table 6 below, by essentially following the procedure as described in Preparation 72, using the appropriate iodopyrazole and boronic acid or ester (1.2-1.5 eq).

TABLE 6

| | | Boronic Acid or | LC-ES/ |
|---|---|---|---|
| Prep | Structure and Chemical Name | Ester | MS (m/z) |
| 73 | Methyl 4-[5-(3-tert-butyl-5- | 2-(3-tert-Butyl-5- | 582 |
| | isopropoxy-phenyl)-1-[4- | isopropoxyphenyl)- | [M + H]⁺ |
| | (morpholine-4- | 4,4,5,5- | |
| | carbonyl)phenyl]pyrazol- | tetramethyl-1,3,2- | |
| | 3-yl]benzoate | dioxaborolane | |
| 74 | Methyl 4-[1-(4-cyanophenyl)-5- | 2,6-Di-tert-butyl- | 493 |
| | (2,6-di-tert-butyl-4- | 4-(4,4,5,5- | [M + H]⁺ |
| | pyridyl)pyrazol-3-yl]benzoate | tetramethyl-1,3,2- | |
| | | dioxaborolan-2- | |
| | | yl)pyridine | |
| 75 | Methyl 4-[5-(3-tert-butyl-5- | (3-tert-Butyl-5- | 524 |
| | isopropyl-phenyl)-1-[4- | isopropyl- | [M + H]⁺ |
| | (dimethylcarbamoyl) | phenyl)boronic | |
| | phenyl]pyrazol- | acid | |
| | 3-yl]benzoate | | |
| 76 | Methyl 4-[5-(2,6-di-tert- | 2,6-Di-tert-butyl- | 539 |
| | butyl-4-pyridyl)-1-[4- | 4-(4,4,5,5- | [M + H]⁺ |
| | (dimethylcarbamoyl)phe- | tetramethyl-1,3,2- | |
| | nyl]pyrazol-3-yl]benzoate | dioxaborolan-2- | |
| | | yl)pyridine | |
| 77 | Methyl 4-[5-(3-tert-butyl-5- | (3-tert-Butyl-5- | 566 |
| | isopropyl-phenyl)-1-[4- | isopropyl- | [M + H]⁺ |
| | (morpholine-4- | phenyl)boronic | |
| | carbonyl)phenyl]pyrazol- | acid | |
| | 3-yl]benzoate | | |
| 78 | Methyl 4-[5-(2,6-di-tert- | 2,6-Di-tert-butyl- | 581 |
| | butyl-4-pyridyl)-1-[4- | 4-(4,4,5,5- | [M + H]⁺ |
| | (morpholine-4-carbonyl)phe- | tetramethyl-1,3,2- | |
| | nyl]pyrazol-3-yl]benzoate | dioxaborolan-2- | |
| | | yl)pyridine | |

### PREPARATION 79

### Methyl 4-[1-(4-carbamoylphenyl)-5-(2,6-di-tert-butyl-4-pyridyl)pyrazol-3-yl]benzoate

Dissolve methyl 4-(5-(3-tert-butyl-5-isopropoxyphenyl)-1-(4-cyanophenyl)-1H-pyrazol-3-yl)benzoate (220 mg, 0.447 mmol) in TFA (4 mL). Add sulfuric acid (1 mL) slowly. Heat the mixture to 45° C. and stir overnight. Pour the mixture into ice water and adjust to about pH=8 with 2 N NaOH (15 mL). Extract the mixture with EtOAc (3×50 mL). Wash the combined organic portions with brine (3×10 mL); dry over Na₂SO₄; filter; collect the filtrate; and concentrate to dryness to afford the title compound (210 mg, 92%) as a white solid. LC-ES/MS m/z 511 [M+H]⁺.

### EXAMPLE 1

### 4-[5-(3,5-Di-tert-butylphenyl)-1-[4-(4-methylpiperazine-1-carbonyl)phenyl]pyrazol-3-yl]benzoic Acid

[chemical structure]

Charge a reactor with THF (1790 L), methyl 4-[5-(3,5-di-tert-butylphenyl)-1-[4-(4-methylpiperazine-1-carbonyl)phenyl]pyrazol-3-yl]benzoate (358 g, 0.605 mol) and water (1140 mL) at 8-12° C. Add lithium hydroxide monohydrate (38 g, 0.905 mol) in one portion. Stir the mixture for 16 h at 8-12° C. Add EtOAc (30 L) and adjust the mixture to pH=5 with 1 N HCl. Separate the two phases and extract the aqueous layer with EtOAc (2×10 L). Combine the organic layers, dry with sodium sulfate, filter, and remove the solvent to give a solid. Purify the material by column chromatography, eluting with DCM/MeOH (20/1) to give the product as a pale yellow solid. Triturate the solid in a solvent mixture of acetonitrile/MTBE (1/3, 4 L) for 1 h. Filter to collect the solid and dry under vacuum at 50° C. for 72 h to give the title compound (264.2 g, 75%) as a white solid. LC-ES/MS m/z 579 [M+H]⁺.

### Crystallization Procedure:

The crystalline free base of 4-[5-(3,5-Di-tert-butylphenyl)-1-[4-(4-methylpiperazine-1-carbonyl)phenyl]pyrazol-3-yl]benzoic acid is prepared by placing 63.6 mg of f4-[5-(3,5-Di-tert-butylphenyl)-1-[4-(4-methylpiperazine-1-carbonyl)phenyl]pyrazol-3-yl]benzoic acid in a 20 mL vial. Add 4 mL of MeOH to prepare a slurry including a white solid. Place the vial with the slurry stirplate heated to 60° C. and stir at 1000 rpm for 2 hours. Thereafter, allow the sample to cool to room temperature. Isolate the resulting white solid by vacuum filtration dry overnight in a vacuum oven set to 70° C. overnight.

### Alternative Crystallization Procedure:

Crystalline 4-[5-(3,5-Di-tert-butylphenyl)-1-[4-(4-methylpiperazine-1-carbonyl)phenyl]pyrazol-3-yl]benzoic acid can also be prepared placing 69 mg of 4-[5-(3,5-Di-tert-butylphenyl)-1-[4-(4-methylpiperazine-1-carbonyl)phenyl]pyrazol-3-yl]benzoic acid and 3 mg of seed crystals of the same form in a 20 mL vial and add 2 mL of MeOH to prepare a slurry containing white solid. The slurry is heated to 60° C. and stirred at 1000 rpm for four hours. Thereafter stop the stirring and allow the sample to cool to room temperature and stand until morning to yield a thick layer of white solid under a clear but slightly yellow supernatant. Isolate the white solid by vacuum filtration and dry under nitrogen stream for 10 minutes before being placed in a new tared vial. This resulting material can be examined by X-Ray Powder Diffraction as described below. Additional this material can be placed in a vacuum oven set to 70° C. to dry completely.

### EXAMPLE 2

### 4-[5-(3,5-Di-tert-butylphenyl)-1-[4-(piperidine-1-carbonyl)phenyl]pyrazol-3-yl]benzoic Acid

[chemical structure]

Dissolve methyl 4-[5-(3,5-di-tert-butylphenyl)-1-[4-(piperidine-1-carbonyl)phenyl]pyrazol-3-yl]benzoate (121 mg, 0.209 mmol) in water (1 mL), THF (3 mL), and methanol (1 mL). Add LiOH (11 mg, 251 mmol). Stir the mixture for about 3 h. Adjust the pH of the mixture with 2 N HCl to pH=7. Dilute the mixture with EtOAc (20 mL). Wash the organics with 2 N HCl (4 mL), and saturated aqueous NaCl (10 mL). Dry the organics over Na₂SO₄, filter, and concentrate under reduced pressure. Purify the crude product by preparatory TLC eluting with 10:1 dichloromethane/methanol to afford the title compound (75 mg, 64%). LC-ES/MS m/z 564 [M+H]⁺.

Prepare the examples in Table 7 below, by essentially following the procedure as described in Example 2, using the appropriate methyl benzoate precursor.

TABLE 7

| | | LC-ES/MS |
|---|---|---|
| Example | Structure and Chemical Name | m/z |
| 3 | [chemical structure] | 496 [M + H]⁺ |
| 4 | [chemical structure] | 524 [M + H]⁺ |
| 5 | [chemical structure] | 554 [M + H]⁺ |
| 6 | [chemical structure] | 582 [M + H]⁺ |
| 7 | [chemical structure] | 566 [M + H]⁺ |
| 8 | [chemical structure] | 567 [M + H]⁺ |
| 9 | [chemical structure] | 510 [M + H]⁺ |
| 10 | [chemical structure] | 525 [M + H]⁺ |
| 11 | [chemical structure] | 552 [M + H]⁺ |

### EXAMPLE 12

### 4-[5-(3-tert-Butyl-5-isopropylsulfanyl-phenyl)-1-[4-(dimethylcarbamoyl)phenyl]pyrazol-3-yl]benzoic Acid

[chemical structure]

Dissolve methyl 4-[5-(3-tert-butyl-5-isopropylsulfanyl-phenyl)-1-[4-(dimethylcarbamoyl)phenyl]pyrazol-3-yl]benzoate (100 mg, 0.180 mmol) in methanol (2 mL) and THF (6 mL). Add 1 M LiOH (2 mL) in a single portion with stirring. Stir the mixture for 3 h at room temperature. Acidify the mixture with 2 N HCl to about pH=2 and extract with EtOAc (3×20 mL). Wash the combined organic portions with brine (2×10 mL), dry over Na₂SO₄, filter and concentrate under reduced pressure to afford the title compound (82 mg, 84%) as a white solid. LC-ES/MS m/z 542 [M+H]⁺.

### EXAMPLE 13

### 4-[5-(3-tert-Butyl-5-isopropylsulfanyl-phenyl)-1-[4-(4-methylpiperazine-1-carbonyl)phenyl]pyrazol-3-yl]benzoic Acid

[chemical structure]

Dissolve methyl 4-[5-(3-tert-butyl-5-isopropylsulfanyl-phenyl)-1-[4-(4-methylpiperazine-1-carbonyl)phenyl]pyrazol-3-yl]benzoate (30 mg, 0.049 mmoles) in THF (3 mL) and methanol (1 mL). Add 1 N lithium hydroxide (1.0 mL, 1.0 mmol). Stir the reaction for 6 h. Adjust to about pH=6 with 1 N HCl. Extract with EtOAc (40 mL). Wash the organic portion with water (2×20 mL), dry over Na₂SO₄, filter, and concentrate to afford the title compound (25 mg, 85%). LC-ES/MS m/z 597 [M+H]⁺.

### EXAMPLE 14

### 4-[5-(3-tert-Butyl-5-isopropoxy-phenyl)-1-[4-(4-methylpiperazine-1-carbonyl)phenyl]pyrazol-3-yl]benzoic Acid

[chemical structure]

Dissolve methyl 4-[5-(3-tert-butyl-5-isopropoxy-phenyl)-1-[4-(4-methylpiperazine-1-carbonyl)phenyl]pyrazol-3-yl]benzoate (0.14 g, 0.235 mmol) in THF (3 mL), methanol (1 mL), and water (1 mL). Add LiOH (0.015 g, 0.353 mmol) and stir for 4 h. Adjust to about pH=7 with 2 N HCl. Separate the layers and wash the organic layer with aqueous NaCl (10 mL). Dry the organics over Na₂SO₄, filter, and concentrate to afford the title compound (135 mg, 99%). LC-ES/MS m/z 581 [M+H]⁺.

### EXAMPLE 15

### 4-[5-(3,5-Di-tert-butylphenyl)-1-(6-methylsulfonyl-3-pyridyl)pyrazol-3-yl]benzoic Acid

[chemical structure]

Dissolve methyl 4-[5-(3,5-di-tert-butylphenyl)-1-(6-methylsulfonyl-3-pyridyl)pyrazol-3-yl]benzoate (102 mg, 0.187 mmol) in water (3 mL), THF (9 mL), and methanol (3 mL). Add LiOH (16 mg, 0.374 mmol) in a single portion. Stir the mixture overnight. Adjust the solution to about pH=2 with 2 N HCl. Extract the mixture 3 times with EtOAc. Wash the combined organics with brine (3×50 mL), dry over Na₂SO₄, filter, and concentrate under reduced pressure. Purify the resulting residue by preparatory TLC to afford the title compound (85 mg, 86%). LC-ES/MS m/z 532 [M+H]⁺.

### EXAMPLE 16

### 4-[5-(3,5-Di-tert-butylphenyl)-1-[6-(dimethylcarbamoyl)-3-pyridyl]pyrazol-3-yl]benzoic Acid

[chemical structure]

Dissolve methyl 4-[5-(3,5-di-tert-butylphenyl)-1-[6-(dimethylcarbamoyl)-3-pyridyl]pyrazol-3-yl]benzoate prep 27 is wrong use prep 44 instead (60 mg, 0.111 mmol) in MeOH (4 mL) and THF (1 mL). Add 1 M LiOH (0.5 mL) in a single portion. Stir the mixture for 5 h at room temperature. Acidify the mixture to about pH=6-7 with 2 N HCl. Extract with EtOAc (50 mL). Wash the organic portion with brine (2×20 mL), dry over Na₂SO₄, filter, and concentrate under reduced pressure. Purify the resulting residue by preparatory TLC eluting with 10:1 DMC/MeOH to afford the title compound (30 mg, 51%). LC-ES/MS m/z 525 [M+H]⁺.

### EXAMPLE 17

### 4-[5-(3,5-Di-tert-butylphenyl)-1-[4-(methanesulfonamido)phenyl]pyrazol-3-yl]benzoic Acid

[chemical structure]

Dissolve methyl 4-[5-(3,5-di-tert-butylphenyl)-1-[4-(methanesulfonamido)phenyl]pyrazol-3-yl]benzoate (230 mg, 411 mmol) in methanol (3 mL) and THF (3 mL). Add 1 M LiOH (1 mL, 1.0 mmol) in a single portion. Stir the mixture overnight. Quench the reaction with dilute aqueous HCl. Extract the mixture 3 times with EtOAc. Dry the combined organic portions over Na₂SO₄, filter, and concentrate to dryness under reduced pressure. Purify the resulting residue by preparatory TLC, eluting with 2:1 EtOAc/petroleum ether to afford the title compound (200 mg, 89%). LC-ES/MS m/z 546 [M+H]⁺.

### EXAMPLE 18

### 4-[5-(3,5-Di-tert-butylphenyl)-1-[4-(dimethylsulfamoyl)phenyl]pyrazol-3-yl]benzoic Acid

[chemical structure]

Dissolve methyl 4-[5-(3,5-di-tert-butylphenyl)-1-[4-(dimethylsulfamoyl)phenyl]pyrazol-3-yl]benzoate (0.11 g, 0.192 mmol) in methanol (1 mL) and THF (6 mL). Add 1 N LiOH (40 mg, 0.953 mmol) in a single portion. Stir the mixture at room temperature overnight. Dilute the reaction mixture with water. Extract with EtOAc (30 mL). Dry the combined organic portions over Na₂SO₄, filter, and concentrate under reduced pressure to afford the title compound (110 mg, 100%). LC-ES/MS m/z 560 [M+H]⁺.

### EXAMPLE 19

### 4-[5-(3,5-Di-tert-butylphenyl)-1-(4-methylsulfonylphenyl)pyrazol-3-yl]benzoic Acid

[chemical structure]

Dissolve methyl 4-[5-(3,5-di-tert-butylphenyl)-1-(4-methylsulfonylphenyl)pyrazol-3-yl]benzoate (58 mg, 0.106 mmol) in ethanol (2.5 mL) and THF (3 mL). Add sodium hydroxide (0.064 mL, 0.319 mmol) at room temperature. Heat the reaction at 50° C. with stirring for 2 h. Cool the reaction to room temperature. Dilute the reaction with water and adjust to about pH=1-2 with 1 N HCl. Stir for 10 min and cool to 4° C. Collect the resulting crystals by vacuum filtration, rinsing with water to afford the title compound (49 mg, 87%) as a white crystalline solid. LC-ES/MS m/z 531 [M+H]⁺.

### EXAMPLE 20

### 4-[5-(3-tert-Butyl-5-isopropoxy-phenyl)-1-(4-carbamoylphenyl)pyrazol-3-yl]benzoic Acid

[chemical structure]

Dissolve methyl 4-[5-(3-tert-butyl-5-isopropoxy-phenyl)-1-(4-carbamoylphenyl)pyrazol-3-yl]benzoate (105 mg, 0.205 mmol) in methanol (6 mL), and THF (2 mL). Add 1 M LiOH (2 mL) in a single portion with stirring. Stir the mixture for 18 h. Add 2 N HCl to pH=6. Extract with EtOAc (50 mL). Wash the organic portion with brine (2×20 mL), dry over Na₂SO₄, filter, and concentrate to dryness under reduced pressure. Purify the crude mixture using flash chromatography on silica (ISCO® system, 12 g cartridge @ 25 mL/min) with a gradient of 0-20% methanol/dichloromethane over 25 min to afford the title compound (93 mg, 91%). LC-ES/MS m/z 498 [M+H]⁺.

### EXAMPLE 21

### 4-[5-(3-tert-Butyl-5-isopropoxy-phenyl)-1-[4-(morpholine-4-carbonyl)phenyl]pyrazol-3-yl]benzoic Acid

[chemical structure]

Dissolve methyl 4-(5-(3-tert-butyl-5-isopropoxyphenyl)-1-(4-(morpholine-4-carbonyl)phenyl)-pyrazol-3-yl)benzoate check prep 73 (160 mg, 0.275 mmol) in methanol (6 mL) and THF (2 mL). Add 1 M LiOH (2 mL) in a single portion and stir overnight. Adjust to about pH=6 with 2 N HCl. Extract with EtOAc (50 mL). Wash the combined organics with brine (2×20 mL), dry over Na₂SO₄, filter, and concentrate to dryness to afford the title compound (136 mg, 87%). LC-ES/MS m/z 568 [M+H]⁺.

### EXAMPLE 22

### 4-[1-(4-Carbamoylphenyl)-5-(2,6-di-tert-butyl-4-pyridyl)pyrazol-3-yl]benzoic Acid

[chemical structure]

Dissolve methyl 4-[1-(4-carbamoylphenyl)-5-(2,6-di-tert-butyl-4-pyridyl)pyrazol-3-yl]benzoate (210 mg, 0.411 mmol) in methanol (6 mL) and THF (2 mL). Add 1 M LiOH (1 mL) in a single portion. Stir the mixture 18 h. Adjust the mixture about pH=6 with 2 N HCl and extract with EtOAc (50 mL). Wash the combined organic portions with brine (2×20 mL), dry over Na₂SO₄, filter, and concentrate to dryness to afford the title compound (180 mg, 88%) as a white solid. LC-ES/MS m/z 497 [M+H]⁺.

### X-Ray Powder Diffraction

The XRD patterns of crystalline 4-[5-(3,5-Di-tert-butylphenyl)-1-[4-(4-methylpiperazine-1-carbonyl)phenyl]pyrazol-3-yl]benzoic acid which can be prepared as described above for Example 1 is obtained on a Bruker D4 Endeavor X-ray powder diffractometer, equipped with a CuKa source λ=1.54060 Å) and a Vantec detector, operating at 35 kV and 50 mA. The sample is scanned between 4 and 40° in 2θ, with a step size of 0.009° in 2θ and a scan rate of 0.5 seconds/step, and with 0.6 mm divergence, 5.28 fixed anti-scatter, and 9.5 mm detector slits. The dry powder is packed on a quartz sample holder and a smooth surface is obtained using a glass slide. The crystal form diffraction patterns are collected at ambient temperature and relative humidity. A peak position variability of ±0.2 in 2θ takes into account potential variations without hindering the unequivocal identification of the indicated crystal form. Confirmation of a crystal form may be made based on any unique combination of distinguishing peaks (in units of ° 2θ), typically the more prominent peaks. The crystal form diffraction pattern, collected at ambient temperature and relative humidity, was adjusted based on NIST 675 standard peaks at 8.853 and 26.774 degrees 2-theta.

Thus, a prepared sample of the free base of 4-[5-(3,5-Di-tert-butylphenyl)-1-[4-(4-methylpiperazine-1-carbonyl)phenyl]pyrazol-3-yl]benzoic acid prepared as described above for Example 1 is characterized by an XRD pattern using CuKa radiation as having diffraction peaks (2-theta values) as described in Table 8 below, and in particular having peaks at 5.414 in combination with one or more of the peaks selected from the group consisting of 19.851, 7.498, and 14.588; with a tolerance for the diffraction angles of 0.2 degrees.

TABLE 8
X-ray powder diffraction peaks of the free base of
4-[5-(3,5-Di-tert-butylphenyl)-1-[4-(4-methylpiperazine-
1-carbonyl)phenyl]pyrazol-3-yl]benzoic acid.

| | Crystalline 4-[5-(3,5-Di-tert-butylphenyl)-1-[4-(4- | | |
|---|---|---|---|
| | methylpiperazine-1-carbonyl)phenyl]pyrazol-3-yl]benzoic acid | | |
| | Peak Positions | | |
| | Angle (°2-Theta) +/− | Relative Intensity | d value |
| Peak | 0.2° | (% of most intense peak) | (angstroms) |
| 1 | 5.4 | 100 | 16.30899 |
| 2 | 19.9 | 49.5 | 4.46887 |
| 3 | 7.5 | 40 | 11.78807 |
| 4 | 14.6 | 37.2 | 6.06742 |
| 5 | 16.0 | 32.5 | 5.52133 |
| 6 | 19.4 | 28.1 | 4.56078 |
| 7 | 15.7 | 27.2 | 5.63154 |
| 8 | 22.1 | 23.9 | 4.01812 |
| 9 | 24.3 | 20.3 | 3.65783 |
| 10 | 18.4 | 17.8 | 4.82536 |

### Assays

The following assay protocols and result(s) thereof demonstrating the utility and efficacy of the compounds and/or methods of the current invention are given for the purpose of illustration and are not meant to be limiting in any way.

### RARα, β and γ Binding Assay

Compounds can be evaluated for binding to RARα, β and γ by measuring their ability to competitively bind to the RAR receptors when dimerized with the binding partner RXRα. Competitive binding assays may be carried out by Scintillation Proximity Assay (SPA) technology using the RARα, β or γ heterodimer (with RXRα as a partner with all the RARs) receptors prepared in a baculovirus expression system. Use the biotinylated oligonucleotide: 5′-ATAATGTAGGTAATAGGTCACCAGGAGGTCAAAGG-3′ for binding of receptor to yttrium silicate streptavidin-coated SPA beads. Per well, preincubate 0.1 nM with 82.7 μg SPA beads in a binding buffer containing 10 mM HEPES pH 7.8, 80 mM KCl, 0.5 mM MgCl₂, 1 mM DTT, 0.5% CHAPS and 16.6 μg bovine serum albumin for 30 min at room temperature. Then spin the mixture at 2,000 rpm for 3 min to pelletize the beads-oligo mix. Remove the supernatant and resuspend the beads-oligo pellet in the same binding buffer as above, but which in addition now also contain 14% glycerol, 5 μg of sheared salmon sperm DNA, 0.5 μg of RARα, 1.0 μg of RARβ, or 0.25 μg of RARγ receptor, respectively. Carry out the binding assays in the presence of ˜11.3 μCi of ³H 4-[(E)-2-(5,6,7,8-tetrahydro-5,5,8,8-tetramethyl-2-naphthalenyl)-1-propenyl]benzoic acid (TTNPB), and multiple concentrations of test compound ranging from (5 nM to 10 μM). Non-specific binding may be determined in the presence of 1 μM unlabeled TTNPB. Use the data to calculate an IC₅₀ for compounds after fitting the dose-response curves to a 4-parameter logistic fit. Use the Cheng-Prusoff equation to convert IC₅₀ (nM) values for compounds to Kᵢ, and the K_{d} may be determined by saturation binding. All of the compounds listed as Examples disclosed herein demonstrate activity in the RARγ binding assay substantially as described herein with a measured Kᵢ of less than 20 nM. All of the compounds listed as Examples disclosed herein demonstrate low activity in the RARβ binding assay substantially as described herein with a measured Kᵢ of greater than 100 nM. All of the compounds listed as Examples disclosed herein exhibit low activity in the RARα binding assay substantially as described herein with a measured Kᵢ of greater than 100 nM. The results of four of the Examples are shown in Table 9 below:

### RARα, β and γ Binding Assay

TABLE 9

| | Kᵢ RARγ | Kᵢ RARα | Kᵢ RARβ |
|---|---|---|---|
| Compound name | (nM) | (nM) | (nM) |
| Example 1 | 1.69 | >970 | >1820 |
| 4-[5-(3,5-Di-tert-butylphenyl)- | n = 1 | n = 1 | n = 1 |
| 1-[4-(4-methylpiperazine-1- | | | |
| carbonyl)phenyl]pyrazol- | | | |
| 3-yl]benzoic acid | | | |
| Example 8 | 1.58 | 357 | 1400 |
| 4-[5-(2,6-Di-tert-butyl-4-pyridyl)- | n = 1 | n = 1 | n = 1 |
| 1-[4-(morpholine-4- | | | |
| carbonyl)phenyl]pyrazol-3- | | | |
| yl]benzoic acid | | | |
| Example 12 | 3.06 | 386 | 609 |
| 4-[5-(3-tert-Butyl-5-isopro- | n = 1 | n = 1 | n = 1 |
| pylsulfanyl-phenyl)- | | | |
| 1-[4-(dimethylcarbamoyl) | | | |
| phenyl]pyrazol-3-yl]benzoic acid | | | |
| Example 19 | 2.76 | 285 | 828 |
| 4-[5-(3,5-Di-tert-butylphenyl)- | n = 1 | n = 1 | n = 1 |
| 1-(4-methylsulfonylphenyl)pyrazol- | | | |
| 3-yl]benzoic acid | | | |

The results of this assay support that the of the Examples disclosed herein bind to the RARγ receptor and the selectivity of the Examples for the RARγ receptor over the RARα, and RARβ receptors.

### Gal4 Reporter Assay to Determine RAR Receptor Antagonist Activity

For cell-based assays, human embryonic kidney HEK 293 cells are transfected with receptor and reporter gene plasmids using Fugene. The reporter plasmid containing five Gal4 binding sites and a major late promoter of adenovirus upstream of the luciferase reporter cDNA is transfected together with a plasmid constitutively expressing the Gal4 DNA binding domain (DBD) and the RARα ligand binding domain (LBD), Gal4 (DBD) RARγ (LBD), or the Gal4 (DBD) RARβ (LBD) hybrid receptor using a SV40 promoter. Cells are transfected in poly-d-lysine coated T175 cm flasks in DMEM media with 5% charcoal-stripped Fetal Bovine Serum (FBS). After an overnight incubation, transfected cells are trypsinized, plated in opaque 96 well dishes in DMEM media containing 5% charcoal-stripped FBS, incubated for 4 h, and then exposed to 0.17 nM to 10 μM of test compound in half log dilutions. To determine the antagonist activity of the test compound, EC₈₀ concentrations of agonist for each receptor is also added to the media (15 nM all-trans retinoic acid, ATRA, for RARα and RARγ, 10 nM of ATRA for RARβ). After 24 hours of incubation with compounds, cells are lysed and luciferase activity is determined. Data are fitted to a four parameter-fit logistics to determine IC₅₀ values. The maximum % inhibition is determined versus the cellular response to 0.25% DMSO in the absence of ATRA. All of the compounds of the Examples disclosed herein demonstrate activity in the Gal4 reporter assay substantially as described herein with a measured K_{b} of less than 250 nM. The results of four of the compounds are shown in Table 10.

TABLE 10
Gal4 Reporter Assay

| Compound name | K_{b} (nM) |
|---|---|
| Example 1 | 23.0 ± 5.36 |
| 4-[5-(3,5-Di-tert-butylphenyl)-1-[4-(4- | n = 2 |
| methylpiperazine-1-carbonyl)phenyl]pyrazol- | |
| 3-yl]benzoic acid | |
| Example 8 | 28.7 |
| 4-[5-(2,6-Di-tert-butyl-4-pyridyl)-1-[4- | n = 1 |
| (morpholine-4-carbonyl)phenyl]pyrazol- | |
| 3-yl]benzoic acid | |
| Example 12 | 30.2 |
| 4-[5-(3-tert-Butyl-5-isopropylsulfanyl-phenyl)- | n = 1 |
| 1-[4-(dimethylcarbamoyl)phenyl]pyrazol- | |
| 3-yl]benzoic acid | |
| Example 19 | 34.6 |
| 4-[5-(3,5-Di-tert-butylphenyl)-1-(4- | n = 1 |
| methylsulfonylphenyl)pyrazol-3-yl]benzoic acid | |

The results of the Gal4 reporter assay support that the Examples disclosed herein are RARγ antagonists.

### RARγ SRC-2 Coactivator Recruitment Assay (Agonist Mode)

The RARγ SRC-2 Coactivator recruitment assay utilizes the ligand binding domain (LBD) of RARγ with its binding partner RXRα to determine the ability of a compound to enhance the recruitment of the co-activator SRC-2 to the receptor complex. Enhanced recruitment of SRC2 is known to be reflective of an agonist confirmation of the RARγ receptor. The RARγ LBD and SRC2 peptides are covalently linked to AlphaScreen® beads such that enhanced protein-protein interactions can be assessed by energy transfer. Coactivator recruitment assays are performed using AlphaScreen® technology (Perkin Elmer USA) using a 6X-Histidine tagged human RARγ LBD and GST tagged hSRC-2 protein. Unlabelled RXRα LBD is added as a silent heterodimer partner. Nickel chelated donor beads are used to bind RARγ LBD and anti-GST acceptor beads are used to bind SRC-2. Serially diluted test compound is added in concentrations ranging from 10 μM to 500 pM to 20 nM human RARγ receptor, 25 nM RXRα LBD, and 5 nM SRC-2 protein in a buffer containing 25 mM HEPES (pH 7.5), 100 mM NaCl, 0.1% Bovine Serum Albumin (fraction V), and 2 mM DTT containing 16.67 μg/ml of nickel chelated donor beads and 16.67 μg/ml of anti GST acceptor beads in a final volume of 15 μl per well in a white 384 shallow well proxiplate. The RAR agonist, TTNPB, is used as a standard on each plate and is added in concentrations ranging from 100 nM to 5 pM. After incubating for 12 hours at room temperature, read the plate on a Perkin Elmer Envision using standard AlphaScreen® parameters for excitation and fluorescence. Use the data to calculate an EC₅₀ for compounds after fitting the dose-response curves to a 4-parameter logistic fit. Calculate the percent stimulation using the fitted top of the TTNPB standard curve as a comparator. All of Examples disclosed herein demonstrate less than 50% maximum stimulation in this assay. The results of four of the compounds are shown in Table 10.

TABLE 11
Percent Stimulation in the RARγ SRC-2
Coactivator Recruitment Assay (Agonist Mode)

| Compound name | % Stimulation |
|---|---|
| Example 1 | 0.2 |
| 4-[5-(3,5-Di-tert-butylphenyl)-1-[4-(4-methylpiperazine- | n = 1 |
| 1-carbonyl)phenyl]pyrazol-3-yl]benzoic acid | |
| Example 8 | 0.6 |
| 4-[5-(2,6-Di-tert-butyl-4-pyridyl)-1-[4-(morpholine-4- | n = 1 |
| carbonyl)phenyl]pyrazol-3-yl]benzoic acid | |
| Example 12 | 8.4 |
| 4-[5-(3-tert-Butyl-5-isopropylsulfanyl-phenyl)-1-[4- | n = 1 |
| (dimethylcarbamoyl)phenyl]pyrazol-3-yl]benzoic acid | |
| Example 19 | −3 |
| 4-[5-(3,5-Di-tert-butylphenyl)-1-(4- | n = 1 |
| methylsulfonylphenyl)pyrazol-3-yl]benzoic acid | |

The results of this assay show that the Examples disclosed herein do not exhibit significant RARγ agonistic activity.

### Monosodium Iodoacetate (MIA) Model of Pain

The injection of monoiodoacetic acid (MIA) into the knee joint of rats produces an acute inflammatory insult, which then develops into chronic degeneration of the joint tissues in the injected joint. The pain resulting from the joint injury can be measured via differential weight bearing of the hind legs using an incapacitance tester. The MIA model has been well-described in the literature and has been used to demonstrate efficacy versus pain for a variety of mechanisms and compounds. Efficacy is routinely measured by the ability of a compound to partially normalize weight distribution. The maximal efficacy that can be achieved in the standard MIA is dependent on the mechanism being studied; however, for many mechanisms the maximal efficacy achieved results in a 25%-50% reduction in the weight bearing differential.

Use Male Lewis rats approximately 150-170 g and between 6-8 weeks of age. Let the animals acclimate to the environment for at least 72 h. Record body weight as needed for dosing schedules and for calibration of Incapacitance Testers. Animals are assigned to treatment groups using the Block Randomized Allocation Tool (BRAT).

MIA sodium salt (from Sigma). Store MIA salt at −80° C. Prepare the MIA, 0.3 mg in 50 μl, in sterile 0.9% saline. Load the syringes with the prepared MIA solution the day the rats are to be injected.

Anesthetize the animals with Isoflurane. Flex the knee joint to locate the joint space between the tibia and the femur. Clean the injection site with 70% ethanol and slowly inject the MIA or saline into the joint space. Inject the right knee with MIA (50 μl) and inject the left knee (contralateral control) with sterile saline (50 μl).

Incapacitance Tester Readings—Incapacitance Testers (Columbus Instruments International, Columbus, Ohio) for weight bearing measurements. Place rats in a plexiglass chamber so that each hind paw rests on a separate force plate (pressure sensor). Allow the rats to acclimate to the chamber for at least 5 minutes. A total of three one second readings are taken to reflect the amount of pressure exerted on both the left and right hind paw while the rat is positioned in the chamber. The force exerted by each hind paw is measured in grams and calculated as the left hind paw weight distribution-right hind paw weight distribution. Thus, the final paw weight distribution for each animal is an average of the three one second readings.

Studies for RARγ antagonists: Dose rats with RARγ antagonist once on day 9 post MIA injection and measure each rat for pain 2 h post dosing. Allow 10-15 min between dosing for each rat to allow 10-15 minutes for pain measurements. Most compounds are initially screened for reduction of pain in a single dose study at 1 or 3 mg/kg of compound before advancing to dose response studies. Generate dose response curves and ED₅₀ values for the RARγ antagonist, by performing either separate dose response studies and combining the results where the 2 studies are: 1) vehicle, RARγ antagonist at 0.1, 0.3 and 1.0 mg/kg, and 2) vehicle, RARγ antagonist at 1, 3, and 10 mg/kg; or as a dose response study where all animals are tested in the same study at 0.1, 0.3, 1.0, 3.0, and 10 mg/kg (Example 1 only). The dose volume for either type of study is 5 ml/kg.

In a dose response study in the standard MIA model, the compound of Example 1 significantly inhibits pain when compared to vehicle at a dose of 0.1 mg/kg.

Exemplified compounds of the present invention can be readily formulated into pharmaceutical compositions in accordance with accepted practice such as found in Remington's Pharmaceutical Sciences, Gennaro, Ed., Mack Publishing Co. Easton Pa. 1990. Oral administration is typically the preferred route of administration for osteoarthritis therapy. Preferred pharmaceutical compositions can be formulated as a tablet or capsule for oral administration. The tablet or capsule can include a compound of the present invention in an effective amount.

The pharmaceutical composition is administered to a patient in amounts effective to treat arthritis, more particularly osteoarthritis and still more preferable for pain associated with osteoarthritis. An appropriate amount or dose effective to treat a patient can be determined by a health care provider and may be dependent upon the age, health, and weight of the recipient, kind of concurrent treatment, if any, frequency of treatment, and the nature of the effect desired. Typical dosage levels can be optimized using standard clinical techniques and will be dependent on the mode of administration and the condition of the patient.

A compound of the present invention can be employed in combination with one or more therapeutic agents, such as, analgesics and/or NSAIDS (nonsteroidal anti inflammatory drug) or COX-2 inhibitors for example, such as aspirin, acetaminophen, celecoxib, diclofenac, ibuprofen, indomethacin, and naproxen, or other anti inflammatory agents.

What is claimed is:

1. A compound having a formula below: [chemical structure]
wherein:
A is CH or N;
X is CH or N;
R1 is selected from: —C(O)N(R3)₂, and —C(O)R4;
R2 is selected from: —C₃₋₄ alkyl, —OCH(CH₃)₂, and —SCH(CH₃)₂;
each R3 is independently selected from: H and —CH₃;
R4 is selected from: 4-morpholinyl, 1-piperidinyl, 4-thiomorpholinyl, —NH(CH₂)₃OH, and 4-methyl-1-piperazinyl; and
provided that when one of A or X is N, the other one of A or X is CH;
or a pharmaceutically acceptable salt thereof.

2. A compound according to claim 1 wherein A is CH.

3. A compound according to claim 1 wherein X is CH.

4. (canceled)

5. A compound according to claim 1 wherein R2 is selected from: —C₃₋₄ alkyl and —SCH(CH₃)₂; or a pharmaceutically acceptable salt thereof.

6. A compound according to claim 1 wherein R2 is selected from: isopropyl, tert-butyl, and —SCH(CH₃)₂, or a pharmaceutically acceptable salt thereof.

7. A compound according to claim 6 wherein R2 is isopropyl or tert-butyl, or a pharmaceutically acceptable salt thereof.

8. A compound according to claim 1 wherein each R3 is —CH₃, or a pharmaceutically acceptable salt thereof.

9. A compound according to claim 1 wherein each R3 is H, or a pharmaceutically acceptable salt thereof.

10. A compound according to claim 1 wherein R4 is selected from: 4-morpholinyl, 1-piperidinyl, 4-thiomorpholinyl, and 4-methyl-1-piperazinyl, or a pharmaceutically acceptable salt thereof.

11. A compound according to claim 10 wherein R4 is 4-morpholinyl or 4-methyl-1-piperazinyl, or a pharmaceutically acceptable salt thereof.

12. A compound according to claim 11 wherein R4 is 4-methyl-1-piperazinyl, or a pharmaceutically acceptable salt thereof.

13. A compound according to claim 1 wherein:
A is CH;
X is CH;
R1 is —C(O)N(R3)₂, or —C(O)R4;
R2 is selected from: —C₃₋₄ alkyl, —OCH(CH₃)₂, and —SCH(CH₃)₂;
each R3 is independently H or CH₃; and
R4 is selected from: 4-morpholinyl, 1-piperidinyl, 4-thiomorpholinyl, —NH(CH₂)₃OH and 4-methyl-1-piperazinyl;
or a pharmaceutically acceptable salt thereof.

14. A compound according to claim 13 wherein,
A is CH;
X is CH;
R1 is —C(O)N(R3)₂, or —C(O)R4;
R2 is selected from: —C₃₋₄ alkyl;
each R3 is independently H or —CH₃; and
R4 is selected from: 4-morpholinyl, 1-piperidinyl, 4-thiomorpholinyl, —NH(CH₂)₃OH and 4-methyl-1-piperazinyl;
or a pharmaceutically acceptable salt thereof.

15. A compound according to claim 1 wherein:
A is CH;
X is N;
R1 is —C(O)N(R3)₂;
R2 is —C₃₋₄ alkyl; and
R3 is H or —CH₃; or
a pharmaceutically acceptable salt thereof.

16. (canceled)

17. A compound according to claim 15 wherein R2 is tert-butyl, or a pharmaceutically acceptable salt thereof.

18. A compound which is 4-[5-(3,5-Di-tert-butylphenyl)-1-[4-(4-methylpiperazine-1-carbonyl)phenyl]pyrazol-3-yl]benzoic acid, or a pharmaceutically acceptable salt thereof.

19. A pharmaceutical composition comprising a compound according to claim 1, or a pharmaceutically acceptable salt thereof, and at least one pharmaceutically acceptable carrier, excipient, or diluent.

20. A pharmaceutical composition comprising a compound as claimed by claim 18 and additionally comprising one or more therapeutic agents.

21. A method of treating osteoarthritic pain in a patent in need of treatment comprising administering to said patient an effective amount of a pharmaceutical composition according to claim 1.

22-24. (canceled)

25. A compound according to formula II [chemical structure]
wherein:
R is selected from C₁₋₄ alkyl, C₁₋₄ haloalkyl, C₃₋₆ cycloalkyl, C₁₋₄ alkyl-C₃₋₆ cycloalkyl, phenyl, and C₁₋₅ alkylphenyl;
A is CH or N;
X is CH or N
R1 is selected from: —C(O)N(R3)₂, and —C(O)R4;
R2 is selected from: —C₃₋₄ alkyl, —OCH(CH₃)₂, and —SCH(CH₃)₂;
each R3 is independently selected from: H and —CH₃;
R4 is selected from: 4-morpholinyl, 1-piperidinyl, 4-thiomorpholinyl, —NH(CH₂)₃OH, and 4-methyl-1-piperazinyl; and
provided that when one of A or X is N, the other one of A or X is CH;
or a pharmaceutically acceptable salt thereof.

26. A process of preparing a compound of formula I or a pharmaceutically acceptable salt thereof, [chemical structure]
wherein:
A is CH or N;
X is CH or N;
R1 is selected from: —C(O)N(R3)₂, and —C(O)R4;
R2 is selected from: —C₃₋₄ alkyl, —OCH(CH₃)₂, and —SCH(CH₃)₂;
each R3 is independently selected from: H and —CH₃;
R4 is selected from: 4-morpholinyl, 1-piperidinyl, 4-thiomorpholinyl, —NH(CH₂)₃OH, and 4-methyl-1-piperazinyl; and
provided that when one of A or X is N, the other one of A or X is CH;
said method comprising de-esterifying a compound of formula II; [chemical structure]
wherein R1 to R4 is as above; and
R is selected from C₁₋₄ alkyl, C₁₋₄ haloalkyl, C₃₋₆ cycloalkyl, C₁₋₄ alkyl-C₃₋₆ cycloalkyl, phenyl, and C₁₋₅ alkylphenyl to provide a compound of formula I, or a pharmaceutically acceptable salt thereof.

27. A compound which is 4-[5-(3,5-Di-tert-butylphenyl)-1-[4-(4-methylpiperazine-1-carbonyl)phenyl]pyrazol-3-yl]benzoic acid in crystalline form characterized by an X-ray powder diffraction pattern obtained from a CuKα source (λ=1.54056 Å) which comprises peaks at:
a) 5.4, 7.5, 14.6, and 19.9+/−0.2 in 2θ; or
b) 5.4, 7.5, 14.6, 16.0, 19.4, and 19.9+/−0.2 in 2θ; or
c) 5.4, 7.5, 14.6, 15.7, 16.0, 19.4, 19.9 and 22.1+/−0.2 in 2θ.
